(19) United States
(12) Patent Application Publication
(10) Pub. No.: US 2025/0049669 A1
(43) Pub. Date: Feb. 13, 2025

(54) COMPOSITION COMPRISING A SPECIFIC OXIDATION DYE PRECURSOR AND TWO SPECIFIC ACIDS

(71) Applicant: L'OREAL, Paris (FR)

(72) Inventors: Anne SABBAGH, SAINT-OUEN (FR); Maxime PAILLARD-BRUNET, SAINT-OUEN (FR)

(21) Appl. No.: 18/704,688

(22) PCT Filed: Oct. 27, 2022
(86) PCT No.: PCT/EP2022/080128

§ 371 Date: Apr. 25, 2024

(30) Foreign Application Priority Data
Oct. 29, 2021 (FR) FR2111574

(51) Int. Cl. A61K 8/41; A61K 8/49; A61K 8/55; A61Q 5/10

(52) U.S. Cl. CPC A61K 8/411; A61K 8/4926; A61K 8/55; A61Q 5/10; A61K 2800/4324

(57) ABSTRACT

The invention relates to a composition comprising at least one oxidation base chosen from 2-methoxymethyl-para-phenylenediamine of formula (I), one of its addition salts, its solvates and/or the solvates of its salts, at least one oxidation coupler, at least one compound chosen from etidronic acid, its salts and their mixtures and at least one compound chosen from 2,6-dipicolinic acid, its salts and their mixtures.

The invention also relates to a method for dyeing keratin fibers, preferably the hair, comprising the application, to said keratin fibers, of the composition.

The invention relates to a composition comprising the combination of a specific oxidation dye precursor with specific acids and/or their salts.

The invention also relates to a method for dyeing keratin fibers, in particular the hair, employing this composition.

Finally, the invention relates to the use of such a composition for dyeing keratin fibers, and in particular the hair.

For a long time, many people have sought to modify the color of their hair and in particular to mask their gray hair.

It is known to dye keratin fibers, in particular human keratin fibers, such as the hair, in order to obtain “permanent” colorings, with dyeing compositions containing oxidation dye precursors, in particular oxidation bases, such as ortho- or para-phenylenediamines, ortho- or para-aminophenols, or heterocyclic compounds, such as pyrazoles, pyrazolinones or pyrazolo-pyridines. These oxidation bases are colorless or weakly colored compounds which, when combined with oxidizing products, can give rise, by an oxidative condensation process, to colored compounds.

It is also possible to vary the shades obtained with these oxidation bases by combining them with couplers or coloring modifiers. The variety of the molecules involved as oxidation bases and couplers makes it possible to obtain a rich palette of colors. However, the use of these dyeing compositions can present a number of disadvantages.

Specifically, after application to keratin fibers, the dyeing power obtained may not be entirely satisfactory, indeed even may be weak, and result in a restricted range of colors.

The colorings may also be insufficiently persistent with respect to external agents, such as light, shampoo or perspiration, and may also be too selective, that is to say that the difference in coloring is too great along one and the same keratin fiber which is differently sensitized between its end and its root.

There exists a real need to make available a composition for dyeing keratin fibers, in particular human keratin fibers, such as the hair, which does not exhibit the abovementioned disadvantages, that is to say which is capable of resulting in a coloring exhibiting an intense coloration with an improved fastness, and also good coverage of the gray hair and good selectivity, and which is capable of resulting in good dyeing performance qualities, even after a period of storage.

These aims and others are achieved by the present invention, a subject matter of which is thus a composition, in particular for the dyeing of keratin fibers, comprising:
at least one oxidation base chosen from 2-methoxymethyl-para-phenylenediamine of formula (I), one of its addition salts, its solvates and/or the solvates of its salts:

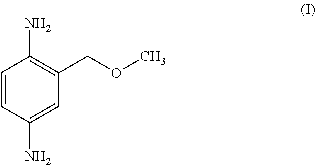

at least one oxidation coupler,
at least one compound chosen from etidronic acid, its salts and their mixtures, and
at least one compound chosen from 2,6-dipicolinic acid, its salts and their mixtures.

Another subject matter of the present invention is a method for dyeing keratin fibers in which the composition according to the invention is applied to said fibers.

According to a preferred embodiment, the composition according to the invention is a composition for the dyeing of keratin fibers, in particular the hair.

The composition according to the invention makes it possible in particular to result in chromatic, powerful, intense and sparingly selective colorings, that is to say colorings which are uniform along the fiber. It also makes it possible to achieve various shades in a very broad palette of colors. In addition, it makes possible a good color buildup.

This composition also makes possible particularly good coverage of depigmented keratin fibers, such as gray hair.

According to the invention, the term “chemical oxidizing agent” is understood to mean an oxidizing agent other than atmospheric oxygen.

Other subject matters, characteristics, aspects and advantages of the invention will become even more clearly apparent on reading the description and the examples which follow.

In that which will follow, unless otherwise indicated, the limits of a range of values are included in that range, in particular in the expressions “of between” and “ranging from . . . to . . . ”.

Moreover, the expression “at least one” used in the present description is equivalent to the expression “one or more”.

The composition according to the invention comprises at least two oxidation dye precursors.

### Oxidation Bases

The composition according to the invention comprises at least one specific oxidation base.

The composition according to the invention comprises at least one oxidation base chosen from 2-methoxymethyl-para-phenylenediamine of formula (I), one of its addition salts, its solvates and/or the solvates of its salts.

The addition salts of the compounds of formula (I) present in the composition according to the invention are chosen in particular from the addition salts with an acid, such as the hydrochlorides, hydrobromides, sulfates, citrates, succinates, tartrates, lactates, tosylates, benzenesulfonates, phosphates and acetates, and the addition salts with a base, such as sodium hydroxide, potassium hydroxide, aqueous ammonia, amines or alkanolamines.

Moreover, the solvates of the compounds of formula (I) more particularly represent the hydrates of said compound and/or the combination of said compound with a linear or branched C₁ to C₄ alcohol, such as methanol, ethanol, isopropanol or n-propanol. Preferably, the solvates are hydrates.

The total content of oxidation base(s) chosen from 2-methoxymethyl-para-phenylenediamine of formula (I), one of its addition salts, its solvates and/or the solvates of its salts preferably varies from 0.001% to 20% by weight, preferably from 0.005% to 15% by weight, more preferentially from 0.01% to 10% by weight, better still from 0.05% to 5%, even better still from 0.1% to 3% by weight, with respect to the total weight of the composition.

The composition according to the invention can optionally in addition comprise one or more additional oxidation bases other than the compounds of formula (I), their addition salts, solvates and/or solvates of their salts chosen from para-phenylenediamines, bis(phenyl)alkylenediamines, para-aminophenols, ortho-aminophenols and heterocyclic bases, their addition salts, their solvates or the solvates of their salts.

Mention may be made, among the para-phenylenediamines other than the compound of formula (I), by way of example, of para-phenylenediamine, para-toluenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene and 3-hydroxy-1-(4′-aminophenyl)pyrrolidine, their addition salts, their solvates and the solvates of their salts.

Mention may be made, among the bis(phenyl)alkylenediamines, by way of example, of N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine, 1,8-bis(2,5-diaminophenoxy)-3,6-dioxaoctane, their addition salts, their solvates and the solvates of their salts.

Mention may be made, among the para-aminophenols, by way of example, of para-aminophenol, 4-amino-3-methylphenol, 4-amino-3-fluorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol or 4-amino-2-fluorophenol, their addition salts, their solvates and the solvates of their salts.

Mention may be made, among the ortho-aminophenols, by way of example, of 2-aminophenol, 2-amino-5-methylphenol, 2-amino-6-methylphenol or 5-acetamido-2-aminophenol, their addition salts, their solvates and the solvates of their salts.

Mention may be made, among the heterocyclic bases, by way of example, of pyridine derivatives, pyrimidine derivatives and pyrazole derivatives.

Mention may be made, among the pyridine derivatives, of the compounds described, for example, in the patents GB 1 026 978 and GB 1 153 196, such as 2,5-diaminopyridine, 2-(4-methoxyphenyl)amino-3-aminopyridine or 3,4-diaminopyridine, their addition salts, their solvates and the solvates of their salts.

Mention may be made, among the pyrimidine derivatives, of the compounds described, for example, in the patents DE 2 359 399; JP 88-169 571; JP 05-63124; EP 0 770 375 or patent application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine or 2,5,6-triaminopyrimidine, and pyrazolopyrimidine derivatives, such as those mentioned in the patent application FR-A-2 750 048, and among which mention may be made of pyrazolo[1,5-a]pyrimidine-3,7-diamine, 2,5-dimethylpyrazolo[1,5-a]pyrimidine-3,7-diamine, pyrazolo[1,5-a]pyrimidine-3,5-diamine, 2,7-dimethylpyrazolo[1,5-a]pyrimidine-3,5-diamine, 3-aminopyrazolo[1,5-a]pyrimidin-7-ol, 3-aminopyrazolo[1,5-a]pyrimidin-5-ol, 2-(3-aminopyrazolo[1,5-a]pyrimidin-7-ylamino)ethanol, 2-(7-aminopyrazolo[1,5-a]pyrimidin-3-ylamino)ethanol, 2-[(3-aminopyrazolo[1,5-a]pyrimidin-7-yl)(2-hydroxyethyl)amino]ethanol, 2-[(7-aminopyrazolo[1,5-a]pyrimidin-3-yl)(2-hydroxyethyl)amino]ethanol, 5,6-dimethylpyrazolo[1,5-a]pyrimidine-3,7-diamine, 2,6-dimethylpyrazolo[1,5-a]pyrimidine-3,7-diamine, 2,5-N7,N7-tetramethylpyrazolo[1,5-a]pyrimidine-3,7-diamine or 3-amino-5-methyl-7-imidazolylpropylaminopyrazolo[1,5-a]pyrimidine, their addition salts, their solvates and the solvates of their salts.

Mention may be made, among the pyrazole derivatives, of diaminopyrazole bases, in particular the compounds described in the patents DE 3843892 and DE 4133957 and patent applications WO 94/08969, WO 94/08970, FR-A-2 733 749 and DE 195 43 988, such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole or 3,5-diamino-4-(β-hydroxyethyl)amino-1-methylpyrazole and the corresponding addition salts. It is also possible to use 4,5-diamino-1-(β-methoxyethyl)pyrazole, their addition salts, their solvates and the solvates of their salts.

The addition salts of the oxidation bases present in the composition according to the invention are in particular chosen from the addition salts with an acid, such as the hydrochlorides, hydrobromides, sulfates, citrates, succinates, tartrates, lactates, tosylates, benzenesulfonates, methanesulfonates, phosphates and acetates, and the addition salts with a base, such as sodium hydroxide, potassium hydroxide, aqueous ammonia, amines or alkanolamines.

Moreover, the solvates of the additional oxidation bases more particularly represent the hydrates of said oxidation bases and/or the combination of said oxidation bases with a linear or branched C₁ to C₄ alcohol, such as methanol, ethanol, isopropanol or n-propanol. Preferably, the solvates are hydrates.

In a specific embodiment, the composition according to the invention is devoid of oxidation bases chosen from para-phenylenediamine, para-toluenediamine, their addition salts, their solvates and the solvates of their salts.

When the composition according to the invention comprises one or more additional oxidation bases, the total content of the additional oxidation base(s), other than 2-(methoxymethyl)benzene-1,4-diamine of formula (I), its addition salts, its solvates and the solvates of its salts, present in the composition according to the invention, preferably varies from 0.001% to 20% by weight, more preferentially from 0.005% to 15% by weight, better still from 0.01% to 10% by weight, even better still from 0.05% to 5% by weight, even better still from 0.1% to 3% by weight, with respect to the total weight of the composition.

### Oxidation Couplers

The composition according to the invention comprises at least one oxidation coupler (or coupling agent) advantageously chosen from those conventionally used in the dyeing of keratin fibers.

Mention may in particular be made, among oxidation couplers, of meta-phenylenediamines, meta-aminophenols, meta-diphenols, naphthalene-based coupling agents and heterocyclic coupling agents, and also the corresponding addition salts.

Mention may be made, for example, of 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, hydroxyethyl-3,4-methylenedioxyaniline, 2-amino-5-ethylphenol, 2,6-bis(β-hydroxyethylamino)toluene, 6-hydroxyindoline, 2,6-dihydroxy-4-methylpyridine, 1H-3-methylpyrazol-5-one, 1-phenyl-3-methylpyrazol-5-one, 2,6-dimethylpyrazolo[1,5-b][1,2,4]triazole, 2,6-dimethyl[3,2-c][1,2,4]triazole, 6-methylpyrazolo[1,5-a]benzimidazole, 2-methyl-5-aminophenol, 5-N-(β-hydroxyethyl)amino-2-methylphenol, 3-aminophenol or 3-amino-2-chloro-6-methylphenol, the corresponding addition salts with an acid and the corresponding mixtures, the solvates, the solvates of their salts and their mixtures.

In general, the addition salts of the couplers which can be used in the context of the invention are chosen in particular from the addition salts with an acid, such as the hydrochlorides, hydrobromides, sulfates, citrates, succinates, tartrates, lactates, tosylates, benzenesulfonates, phosphates and acetates.

According to a preferred embodiment, the composition according to the present invention comprises one or more couplers chosen from: 6-hydroxybenzomorpholine of formula (II), its addition salts, its solvates and/or the solvates of its salts, hydroxyethyl-3,4-methylenedioxyaniline of formula (III), its addition salts, its solvates and/or the solvates of its salts, 2-amino-5-ethylphenol of formula (IV), its addition salts, its solvates and/or the solvates of its salts, and their mixtures.

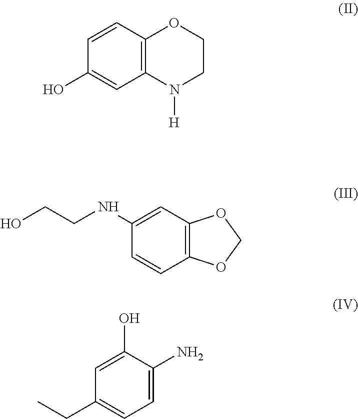

The addition salts of the compounds of formulae (II), (III) and (IV) are chosen in particular from the addition salts with an acid, such as the hydrochlorides, hydrobromides, sulfates, citrates, succinates, tartrates, lactates, tosylates, benzenesulfonates, phosphates and acetates, and the addition salts with a base, such as sodium hydroxide, potassium hydroxide, aqueous ammonia, amines or alkanolamines.

In a specific embodiment, the composition according to the invention is devoid of oxidation couplers chosen from resorcinol, 2-methylresorcinol, 4-chlororesorcinol, their addition salts, their solvates and the solvates of their salts.

Moreover, the solvates of the compounds of formulae (II), (III) and (IV) more particularly represent the hydrates of these compounds and/or the combination of these compounds with a linear or branched C₁ to C₄ alcohol, such as methanol, ethanol, isopropanol or n-propanol. Preferably, the solvates are hydrates.

The total content of the coupler(s) preferably varies from 0.001% to 20% by weight, more preferentially from 0.005% to 15% by weight, better still from 0.01% to 10% by weight, even better still from 0.05% to 5% by weight, even better still from 0.1% to 3% by weight, with respect to the total weight of the composition.

When the composition comprises one or more oxidation couplers chosen from the couplers of formulae (II), (III) and (IV) and also their addition salts, their solvates and/or the solvates of their salts, their total content preferably varies from 0.001% to 20% by weight, more preferentially from 0.005% to 15% by weight, better still from 0.01% to 10% by weight, even better still from 0.05% to 5% by weight, even better still from 0.1% to 3% by weight, with respect to the total weight of the composition.

### 2,6-Dipicolinic Acid, its Salts

The composition according to the invention also comprises one or more compounds chosen from 2,6-dipicolinic acid, its salts and their mixtures.

The salts are in particular alkali metal, alkaline earth metal, ammonium and substituted ammonium salts.

Preference is given, among the salts of these compounds, to the alkali metal salts and in particular the sodium or potassium salts.

The composition according to the invention preferably comprises 2,6-dipicolinic acid (INCI name: 2,6-dicarboxypyridine).

Preferably, the total content of compound(s) chosen from 2,6-dipicolinic acid, its salts and their mixtures ranges from 0.001% to 15% by weight, more preferentially from 0.005% to 10% by weight, better still from 0.01% to 8% by weight, even better still from 0.05% to 5% by weight, indeed even from 0.075% to 2% by weight, with respect to the total weight of the composition.

According to a preferred embodiment, the total content of 2,6-dipicolinic acid ranges from 0.001% to 15% by weight, more preferentially from 0.005% to 10% by weight, better still from 0.01% to 8% by weight, even better still from 0.05% to 5% by weight, indeed even from 0.075% to 2% by weight, with respect to the total weight of the composition.

### Etidronic Acid, its Salts

The composition according to the invention also comprises one or more compounds chosen from etidronic acid, its salts and their mixtures.

The salts are in particular alkali metal, alkaline earth metal, ammonium and substituted ammonium salts.

Preference is given, among the salts of these compounds, to the alkali metal salts and in particular the sodium or potassium salts.

The etidronic acid salts can be chosen from tetrasodium etidronate, disodium etidronate, tetrapotassium etidronate, dipotassium etidronate and a mixture of these compounds.

The composition according to the invention preferably comprises etidronic acid. Use will be made, for example, of Turpinal SL from Talmatch Chemicals.

Preferably, the total content of compound(s) chosen from etidronic acid, its salts and their mixtures ranges from 0.001% to 15% by weight, more preferentially from 0.005% to 10% by weight, better still from 0.01% to 8% by weight, even better still from 0.05% to 5% by weight, indeed even from 0.075% to 2% by weight, with respect to the total weight of the composition.

According to a preferred embodiment, the total content of etidronic acid ranges from 0.001% to 15% by weight, more preferentially from 0.005% to 10% by weight, better still from 0.01% to 8% by weight, even better still from 0.05% to 5% by weight, indeed even from 0.075% to 2% by weight, with respect to the total weight of the composition.

### Surfactants

The composition according to the present invention can comprise one or more surfactants. These surfactants can be chosen from anionic surfactants, amphoteric surfactants, nonionic surfactants, cationic surfactants and/or their mixtures.

The term “anionic surfactant” is understood to mean a surfactant comprising, as ionic or ionizable groups, only anionic groups. These anionic groups are preferably chosen from the following groups: CO₂H, CO₂⁻, SO₃H, SO₃⁻, OSO₃H, OSO₃⁻, H₂PO₃, HPO₃⁻, PO₃²⁻, H₂PO₂, HPO₂⁻, PO₂²⁻, POH and PO⁻.

Mention may be made, as examples of anionic surfactants which can be used in the composition according to the invention, of alkyl sulfates, alkyl ether sulfates, alkylamido ether sulfates, alkylaryl polyether sulfates, monoglyceride sulfates, alkylsulfonates, alkylamidesulfonates, alkylarylsulfonates, α-olefinsulfonates, paraffin sulfonates, alkyl sulfosuccinates, alkyl ether sulfosuccinates, alkylamide sulfosuccinates, alkyl sulfoacetates, acyl sarcosinates, acyl glutamates, alkyl sulfosuccinamates, acyl isethionates and N—(C₁-C₄)alkyl-N-acyltaurates, salts of alkyl monoesters of polyglycoside-polycarboxylic acids, acyl lactylates, salts of D-galactoside uronic acids, salts of alkyl ether carboxylic acids, salts of alkylaryl ether carboxylic acids, salts of alkylamido ether carboxylic acids; and the corresponding nonsalified forms of all these compounds; the alkyl and acyl groups of all these compounds (unless specified otherwise) generally comprising from 6 to 24 carbon atoms and the aryl group generally denoting a phenyl group.

These compounds can be oxyethylenated and then preferably comprise from 1 to 50 ethylene oxide units.

The salts of C₆-C₂₄ alkyl monoesters and of polyglycoside-polycarboxylic acids can be chosen from C₆-C₂₄ alkyl polyglycoside-citrates, C₆-C₂₄ alkyl polyglycoside-tartrates and C₆-C₂₄ alkyl polyglycoside-sulfosuccinates. When the anionic surfactant(s) are in the salt form, they can be chosen from alkali metal salts, such as the sodium or potassium and preferably sodium salt, ammonium salts, amine salts and in particular aminoalcohol salts, or alkaline earth metal salts, such as the magnesium salt.

Mention may in particular be made, as examples of aminoalcohol salts, of monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

Use is preferably made of alkali metal salts or alkaline earth metal salts and in particular the sodium or magnesium salts.

The anionic surfactants optionally present can be mild anionic surfactants, that is to say without a sulfate function.

As regards the mild anionic surfactants, mention may in particular be made of the following compounds and their salts, and also their mixtures: polyoxyalkylenated alkyl ether carboxylic acids, polyoxyalkylenated alkylaryl ether carboxylic acids, polyoxyalkylenated alkylamido ether carboxylic acids, in particular those comprising from 2 to 50 ethylene oxide groups, alkyl D-galactoside uronic acids, acyl sarcosinates, acyl glutamates and alkylpolyglycoside carboxylic esters.

Very particularly, use may be made of polyoxyalkylenated alkyl ether carboxylic acids, such as, for sample, lauryl ether carboxylic acid (4.5 OE), for example sold under the name Akypo RLM 45 CA from Kao.

Use is preferably made, among the abovementioned anionic surfactants, of sulfated surfactants, such as alkyl sulfates or alkyl ether sulfates, and acyl glutamates, more preferentially alkyl sulfates and/or alkyl ether sulfates.

The amphoteric or zwitterionic surfactant(s) which can be used in the composition according to the invention are preferably nonsilicone surfactants and can in particular be optionally quaternized secondary or tertiary aliphatic amine derivatives, in which the aliphatic group is a linear or branched chain comprising from 8 to 22 carbon atoms, said amine derivatives containing at least one anionic group, such as, for example, a carboxylate, sulfonate, sulfate, phosphate or phosphonate group.

Mention may in particular be made of (C₈-C₂₀)alkyl betaines, (C₈-C₂₀)alkyl sulfobetaines, (C₈-C₂₀)alkylamido(C₁-C₆)alkyl betaines, (C₈-C₂₀)alkylamido(C₁-C₆)alkyl sulfobetaines, and their mixtures.

Mention may also be made, among optionally quaternized secondary or tertiary aliphatic amine derivatives which can be used, as defined above, of the compounds with the following respective structures (VI) and (VII):

Rₐ—CONHCH₂CH₂—N⁺(R_{b})(R_{c})—CH₂COO⁻,M⁺,X⁻ (VI)

in which formula (VI):
Rₐ represents a C₁₀ to C₃₀ alkyl or alkenyl group derived from an acid RₐCOOH preferably present in hydrolyzed copra oil; preferably, Rₐ represents a heptyl, nonyl or undecyl group;
R_{b} represents a β-hydroxyethyl group;
Rₑ represents a carboxymethyl group;
M⁺ represents a cationic counterion resulting from an alkali metal or alkaline earth metal, such as sodium, an ammonium ion or an ion resulting from an organic amine; and
X⁻ represents an organic or inorganic anionic counterion, such as that chosen from halides, acetates, phosphates, nitrates, (C₁-C₄)alkyl sulfates, (C₁-C₄)alkylsulfonates or (C₁-C₄)alkylarylsulfonates, in particular methyl sulfate and ethyl sulfate; or alternatively M⁺ and X⁻ are absent;

Rₐ′—CONHCH₂CH₂—N(B)(B′) (VII)

in which formula (VII):
B represents the group —CH₂CH₂OX′;
B′ represents the group —(CH₂)_{z}Y′, with z=1 or 2;
X′ represents the group —CH₂COOH, —CH₂—COOZ′, —CH₂CH₂COOH or CH₂CH₂—COOZ′, or a hydrogen atom;
Y′ represents the group —COOH, —COOZ′ or —CH₂CH(OH)SO₃H or the group CH₂CH(OH)SO₃—Z′;
Z′ represents a cationic counterion resulting from an alkali metal or alkaline earth metal, such as sodium, an ammonium ion or an ion resulting from an organic amine;
Rₐ′ represents a C₁₀ to C₃₀ alkyl or alkenyl group of an acid Rₐ′—COOH preferably present in hydrolyzed linseed oil or copra oil; preferably, Rₐ′ is an alkyl group, in particular a C₁₇ group, and its iso form, or an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

Mention may be made, by way of example, of the cocoamphodiacetate sold by Rhodia under the trade name Miranol® C2M Concentrate.

Use may also be made of the compounds of formula (VIII):

Rₐ″—NHCH(Y″)—(CH₂)ₙCONH(CH₂)_{n′}—N(R_{d})(Rₑ) (VIII)

in which formula (VIII):
Y″ represents the group —COOH, —COOZ″ or —CH₂—CH(OH)SO₃H or the group CH₂CH(OH)SO₃—Z″;
R_{d} and Rₑ represent, independently of each other, a C₁ to C₄ alkyl or hydroxyalkyl radical;
Z″ represents a cationic counterion resulting from an alkali metal or alkaline earth metal, such as sodium, an ammonium ion or an ion resulting from an organic amine;
Rₐ″ represents a C₁₀ to C₃₀ alkyl or alkenyl group of an acid Rₐ″—COOH preferably present in hydrolyzed linseed oil or copra oil; and
n and n′ denote, independently of each other, an integer ranging from 1 to 3.

Mention may be made, among the compounds of formula (V), of the compound classified in the CTFA dictionary under the name sodium diethylaminopropyl cocoaspartamide and sold by Chimex under the name Chimexane HB.

These compounds can be used alone or as mixtures.

Use is advantageously made, among the abovementioned amphoteric or zwitterionic surfactants, of (C₈-C₂₀)alkyl betaines, such as coco betaine, (C₈-C₂₀)alkylamido(C₃-C₈)alkyl betaines, such as cocamidopropyl betaine, (C₈-C₂₀)alkylamphoacetates, (C₈-C₂₀)alkylamphodiacetates and their mixtures; and preferably (C₈-C₂₀)alkyl betaines, (C₈-C₂₀)alkylamido(C₃-C₈)alkyl betaines and their mixtures.

Preferentially, the amphoteric or zwitterionic surfactant(s) are chosen from (C₈-C₂₀)alkyl betaines, (C₈-C₂₀)alkylamido(C₃-C₈)alkyl betaines and their mixtures.

The nonionic surfactant(s) which can be used in the composition of the present invention are in particular described, for example, in the Handbook of Surfactants by M. R. Porter, published by Blackie & Son (Glasgow and London), 1991, pages 116-178.

Mention may be made, as examples of nonionic surfactants, of the following compounds, alone or as a mixture:
oxyalkylenated (C₈-C₂₄)alkylphenols;
saturated or unsaturated, linear or branched, oxyalkylenated or glycerolated, C₈ to C₄₀ alcohols; they preferably comprise one or two fatty chains;
saturated or unsaturated, linear or branched, oxyalkylenated C₈-C₃₀ fatty acid amides;
esters of saturated or unsaturated, linear or branched, C₈-C₃₀ fatty acids and of glycerol;
esters of saturated or unsaturated, linear or branched, C₈ to C₃₀ acids and of polyethylene glycols;
esters of fatty acids and of sucrose;
esters of saturated or unsaturated, linear or branched, C₈ to C₃₀ acids and of sorbitol which are preferably oxyethylenated;
C₈-C₃₀ fatty acid esters of sorbitan,
polyoxyethylenated C₈-C₃₀ fatty acid esters of sorbitan,
(C₈-C₃₀)alkyl(poly)glucosides, (C₈-C₃₀)alkenyl(poly)glucosides, which are optionally oxyalkylenated (0 to 10 oxyalkylene units) and which comprise from 1 to 15 glucose units, (C₈-C₃₀)alkyl(poly)glucoside esters;
saturated or unsaturated oxyethylenated vegetable oils;
condensates of ethylene oxide and/or of propylene oxide;
N—(C₈-C₃₀)alkylglucamine and N—(C₈-C₃₀)acylmethylglucamine derivatives;
amine oxides.

They are chosen in particular from alcohols, α-diols or (C₁-C₂₀)alkylphenols, these compounds being ethoxylated, propoxylated or glycerolated and having at least one fatty chain comprising, for example, from 8 to 24 carbon atoms, preferably from 8 to 18 carbon atoms, it being possible for the number of ethylene oxide or propylene oxide groups to range in particular from 1 to 200 and for the number of glycerol groups to range in particular from 1 to 30.

Mention may also be made of condensates of ethylene oxide and of propylene oxide with fatty alcohols; ethoxylated fatty amides preferably having from 1 to 30 ethylene oxide units, polyglycerolated fatty amides comprising on average from 1 to 5 and in particular from 1.5 to 4 glycerol groups, fatty acid esters of sucrose, fatty acid esters of polyethylene glycol, oxyethylenated vegetable oils, N—(C₆-C₂₄ alkyl)glucamine derivatives, amine oxides, such as (C₁₀-C₁₄)alkylamine oxides or N—(C₁₀-C₁₄ acyl)aminopropylmorpholine oxides.

The esters of saturated or unsaturated, linear or branched, C₈-C₃₀ fatty acids and of glycerol can be chosen from esters of C₈-C₂₄, preferably C₁₂-C₂₂, preferentially C₁₄-C₂₀, fatty acid(s) and of glycerol.

The C₈-C₃₀ fatty acid ester(s) of glycerol can be chosen from monoesters, diesters and their mixtures, preferably from the monoesters.

Preferably, the ester(s) of C₈-C₃₀ fatty acids are chosen from esters of saturated linear C₁₂-C₂₂, preferentially C₁₄-C₂₀, fatty acid(s) and of glycerol, more preferentially from monoesters of saturated linear C₁₂-C₂₂, preferentially C₁₄-C₂₀, fatty acid(s) and of glycerol.

Use will more particularly be made of glycerol monostearate. Mention may be made, by way of examples of glyceryl monostearate, of the following commercial products: Cutina GMS V and Cutina MD from Cognis; Lipo GMS 450 V from Lipo Chemicals; Tegin ISO from Goldschmidt; Lexemul 55 G from Inolex; Witconol MST from CK Witco; Tegin 6070 from Evonik Goldschmidt; Tegin 515, Tegin 90 Pellets and Tegin M Pellets from Evonik Goldschmidt; Estol 1473 from Croda; DUB GMS 50/50 from Stearinerie Dubois.

The C₈-C₃₀, preferably C₁₂-C₂₂, fatty acid esters (in particular monoesters, diesters, triesters) of sorbitan can be chosen from:
sorbitan caprylate; sorbitan cocoate; sorbitan isostearate; sorbitan laurate; sorbitan oleate; sorbitan palmitate; sorbitan stearate; sorbitan diisostearate; sorbitan dioleate; sorbitan distearate; sorbitan sesquicaprylate; sorbitan sesquiisostearate; sorbitan sesquioleate; sorbitan sesquistearate; sorbitan triisostearate; sorbitan trioleate; or sorbitan tristearate.

The esters (in particular monoesters, diesters, triesters) of C₈-C₃₀ fatty acids and of polyoxyethylenated sorbitan are preferably chosen from C₈-C₃₀ fatty acid ester(s) of oxyethylenated sorbitan having from 1 to 30 ethylene oxide units, preferably from 2 to 20 ethylene oxide units, more preferably from 2 to 10 ethylene oxide units.

Preferentially, the C₈-C₃₀ fatty acid ester(s) of oxyethylenated sorbitan is/are chosen from esters of C₁₂-C₁₈ fatty acids and of oxyethylenated sorbitan, in particular from oxyethylenated esters of lauric acid, of myristic acid, of cetylic acid and of stearic acid and of sorbitan.

Preferably, the C₈-C₃₀ fatty acid ester(s) of oxyethylenated sorbitan is/are chosen from oxyethylenated sorbitan monolaurate (4 OE) (Polysorbate-21), oxyethylenated sorbitan monolaurate (20 OE) (Polysorbate-20), oxyethylenated sorbitan monopalmitate (20 OE) (Polysorbate-40), oxyethylenated sorbitan monostearate (20 OE) (Polysorbate-60), oxyethylenated sorbitan monostearate (4 OE) (Polysorbate-61), oxyethylenated sorbitan monooleate (20 OE) (Polysorbate-80), oxyethylenated sorbitan monooleate (5 OE) (Polysorbate-81), oxyethylenated sorbitan tristearate (20 OE) (Polysorbate-65), oxyethylenated sorbitan trioleate (20 OE) (Polysorbate-85).

The nonionic surfactant(s) are preferably chosen from ethoxylated C₈-C₂₄ fatty alcohols comprising from 1 to 200 ethylene oxide groups, (C₆-C₂₄ alkyl)polyglycosides, esters of saturated or unsaturated, linear or branched, C₈-C₃₀ fatty acids and of glycerol, esters of C₈-C₃₀ fatty acids and of oxyethylenated sorbitan, and their mixtures, preferentially from ethoxylated C₈-C₂₄ fatty alcohols comprising from 1 to 200 ethylene oxide groups, (C₆-C₂₄ alkyl)polyglycosides, esters of saturated or unsaturated, linear or branched, C₈-C₃₀ fatty acids and of glycerol.

The cationic surfactant(s) which can be used in the composition according to the invention are generally chosen from optionally polyoxyalkylenated primary, secondary or tertiary fatty amines, quaternary ammonium salts, and their mixtures.

The fatty amines generally comprise at least one C₈-C₃₀ hydrocarbon chain. Mention may be made, among the fatty amines which can be used according to the invention, for example, of stearylamidopropyldimethylamine and distearylamine.

Mention may in particular be made, as quaternary ammonium salts, for example, of:
those corresponding to the following general formula (X):

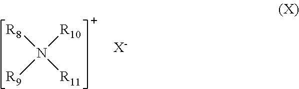

in which the groups R₈ to R₁₁, which can be identical or different, represent a linear or branched aliphatic group comprising from 1 to 30 carbon atoms, or an aromatic group, such as aryl or alkylaryl, at least one of the groups R₈ to R₁₁ comprising from 8 to 30 carbon atoms, preferably from 12 to 24 carbon atoms. The aliphatic groups can comprise heteroatoms, such as in particular oxygen, nitrogen, sulfur and halogens.

The aliphatic groups are chosen, for example, from C₁-C₃₀ alkyl, C₁-C₃₀ alkoxy, polyoxy(C₂-C₆)alkylene, C₁-C₃₀ alkylamide, (C₁₂-C₂₂)alkylamido(C₂-C₆)alkyl, (C₁₂-C₂₂)alkyl acetate and C₁-C₃₀ hydroxyalkyl groups; X⁻ is an anion chosen from the group of the halides, phosphates, acetates, lactates, (C₁-C₄)alkyl sulfates, (C₁-C₄)alkylsulfonates and (C₁-C₄)alkylarylsulfonates.

Preference is given, among the quaternary ammonium salts of formula (X), first, to tetraalkylammonium chlorides, such as, for example, dialkyldimethylammonium or alkyltrimethylammonium chlorides in which the alkyl group comprises approximately from 12 to 22 carbon atoms, in particular behenyltrimethylammonium chloride, distearyldimethylammonium chloride, cetyltrimethylammonium chloride or benzyldimethylstearylammonium chloride, or also, secondly, to distearoylethylhydroxyethylmethylammonium methosulfate, dipalmitoylethylhydroxyethylammonium methosulfate or distearoylethylhydroxyethylammonium methosulfate, or also, finally, to palmitylamidopropyltrimethylammonium chloride or stearamidopropyldimethyl(myristyl acetate)ammonium chloride, sold under the name Ceraphyl® 70 by Van Dyk;
quaternary ammonium salts of imidazoline, such as, for example, those of the following formula (XI):

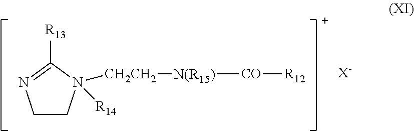

in which R₁₂ represents an alkenyl or alkyl group comprising from 8 to 30 carbon atoms, for example derived from tallow fatty acids, R₁₃ represents a hydrogen atom, a C₁-C₄ alkyl group or an alkenyl or alkyl group comprising from 8 to 30 carbon atoms, R₁₄ represents a C₁-C₄ alkyl group, R₁₅ represents a hydrogen atom or a C₁-C₄ alkyl group and X⁻ is an anion chosen from the group of the halides, phosphates, acetates, lactates, (C₁-C₄)alkyl sulfates, (C₁-C₄)alkylsulfonates and (C₁-C₄)alkylarylsulfonates.

Preferably, R₁₂ and R₁₃ denote a mixture of alkenyl or alkyl groups comprising from 12 to 21 carbon atoms, for example derived from tallow fatty acids, R₁₄ denotes a methyl group and R₁₅ denotes a hydrogen atom. Such a product is sold, for example, under the name Rewoquat® W 75 by Rewo,
di- or diquaternary ammonium salts, in particular of the following formula (XII):

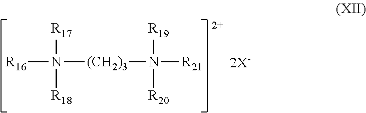

in which R₁₆ denotes an alkyl group comprising approximately from 16 to 30 carbon atoms which is optionally hydroxylated and/or interrupted by one or more oxygen atoms; R₁₇ is chosen from hydrogen, an alkyl group comprising from 1 to 4 carbon atoms or a group —(CH₂)₃—N⁺(R₁₆ₐ)(R₁₇ₐ)(R₁₈ₐ), R₁₆ₐ, R₁₇ₐ, R₁₈ₐ, R₁₈, R₁₉, R₂₀ and R₂₁, which are identical or different, are chosen from hydrogen or an alkyl group comprising from 1 to 4 carbon atoms, and X⁻ is an anion chosen from the group of the halides, acetates, phosphates, nitrates, (C₁-C₄)alkyl sulfates, (C₁-C₄)alkylsulfonates and (C₁-C₄)alkylarylsulfonates, in particular methyl sulfate and ethyl sulfate.

Such compounds are, for example, Finquat CT-P, provided by Finetex (Quaternium 89), or Finquat CT, provided by Finetex (Quaternium 75);
quaternary ammonium salts containing one or more ester functions, such as, for example, those of the following formula (XIII):

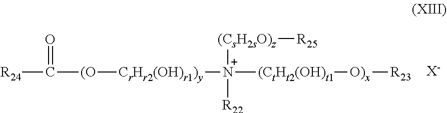

in which: R₂₂ is chosen from C₁-C₆ alkyl groups and C₁-C₆ hydroxyalkyl or dihydroxyalkyl groups; R₂₃ is chosen from: the group —C(O)R₂₆, saturated or unsaturated, linear or branched, C₁-C₂₂ hydrocarbon groups R₂₇ or a hydrogen atom; R₂₅ is chosen from: the group —C(O)R₂₈, saturated or unsaturated, linear or branched, C₁-C₆ hydrocarbon groups R₂₉ or a hydrogen atom; R₂₄, R₂₆ and R₂₈, which are identical or different, are chosen from saturated or unsaturated, linear or branched, C₇-C₂₁ hydrocarbon groups; r, s and t, which are identical or different, are integers having values from 2 to 6; r1 and t1, which are identical or different, have the values 0 or 1; r2+r1=2 r and t1+t2=2 t, y is an integer having values from 1 to 10, x and z, which are identical or different, are integers having values from 0 to 10, X⁻ is a simple or complex organic or inorganic anion, with the proviso that the sum x+y+z has a value from 1 to 15, that, when x has the value 0, then R₂₃ denotes R₂₇ and that, when z has the value 0, then R₂₅ denotes R₂₉.

The alkyl groups R₂₂ can be linear or branched and more particularly linear.

Preferably, R₂₂ denotes a methyl, ethyl, hydroxyethyl or dihydroxypropyl group and more particularly a methyl or ethyl group.

Advantageously, the sum x+y+z has a value from 1 to 10.

When R₂₃ is a hydrocarbon group R₂₇, it can be long and have from 12 to 22 carbon atoms or short and have from 1 to 3 carbon atoms.

When R₂₅ is a hydrocarbon group R₂₉, it preferably has from 1 to 3 carbon atoms.

Advantageously, R₂₄, R₂₆ and R₂₈, which are identical or different, are chosen from saturated or unsaturated, linear or branched, C₁₁-C₂₁ hydrocarbon groups, and more particularly from saturated or unsaturated, linear or branched, C₁₁-C₂₁ alkyl and alkenyl groups.

Preferably, x and z, which are identical or different, have the values 0 or 1.

Advantageously, y is equal to 1.

Preferably, r, s and t, which are identical or different, have the values 2 or 3 and more particularly still are equal to 2.

The anion X⁻ is preferably a halide, preferably chloride, bromide or iodide, a (C₁-C₄)alkyl sulfate, a (C₁-C₄)alkylsulfonate or a (C₁-C₄)alkylarylsulfonate. However, use may be made of methanesulfonate, phosphate, nitrate, tosylate, an anion derived from an organic acid, such as acetate or lactate, or any other anion compatible with the ammonium having an ester function.

The anion X⁻ is more particularly still chloride, methyl sulfate or ethyl sulfate.

Use is made more particularly, in the composition according to the invention, of the ammonium salts of formula (XIII) in which: R₂₂ denotes a methyl or ethyl group, x and y are equal to 1, z is equal to 0 or 1, r, s and t are equal to 2; R₂₃ is chosen from: the group —C(O)R₂₆, methyl, ethyl or C₁₄-C₂₂ hydrocarbon groups or a hydrogen atom, R₂₅ is chosen from: the group —C(O)R₂₈ or a hydrogen atom, R₂₄, R₂₆ and R₂₈, which are identical or different, are chosen from saturated or unsaturated, linear or branched, C₁₃-C₁₇ hydrocarbon groups and preferably from saturated or unsaturated, linear or branched, C₁₃-C₁₇ alkyl and alkenyl groups.

Advantageously, the hydrocarbon groups are linear.

Mention may be made, for example, among the compounds of formula (XIII), of the salts, in particular the chloride or methyl sulfate, of diacyloxyethyldimethylammonium, of diacyloxyethylhydroxyethylmethylammonium, of monoacyloxyethyldihydroxyethylmethylammonium, of triacyloxyethylmethylammonium or of monoacyloxyethylhydroxyethyldimethylammonium, and their mixtures. The acyl groups preferably contain 14 to 18 carbon atoms and are derived more particularly from a plant oil such as palm oil or sunflower oil. When the compound contains several acyl groups, these groups may be identical or different.

These products are obtained, for example, by direct esterification of triethanolamine, triisopropanolamine, an alkyldiethanolamine or an alkyldiisopropanolamine, which are optionally oxyalkylenated, with fatty acids or with fatty acid mixtures of plant or animal origin, or by transesterification of the methyl esters thereof. This esterification is followed by quaternization by means of an alkylating agent such as an alkyl halide, preferably methyl or ethyl halide, a dialkyl sulfate, preferably dimethyl or diethyl sulfate, methyl methanesulfonate, methyl para-toluenesulfonate, glycol chlorohydrin or glycerol chlorohydrin.

Such compounds are sold, for example, under the names Dehyquart® by the company Henkel, Stepanquat® by the company Stepan, Noxamium® by the company CECA or Rewoquat® WE 18 by the company Rewo-Witco.

The composition according to the invention may contain, for example, a mixture of quaternary ammonium monoester, diester and triester salts with a weight majority of diester salts.

Use may also be made of the ammonium salts containing at least one ester function that are described in patents U.S. Pat. Nos. 4,874,554 and 4,137,180.

Use may also be made of the behenoylhydroxypropyltrimethylammonium chloride sold, for example, by the company Kao under the name Quartamin BTC 131.

Preferably, the ammonium salts containing at least one ester function contain two ester functions.

Among the cationic surfactants, it is more particularly preferred to choose cetyltrimethylammonium, behenyltrimethylammonium and dipalmitoylethylhydroxyethyl-methylammonium salts, and mixtures thereof, and more particularly behenyltrimethylammonium chloride, cetyltrimethylammonium chloride, and dipalmitoylethylhydroxyethylammonium methosulfate, and mixtures thereof.

Preferably, the surfactant(s) are chosen from anionic surfactants, nonionic surfactants, amphoteric surfactants and their mixtures.

More preferentially, the surfactant(s) are chosen from nonionic surfactants, better still from ethoxylated C₈-C₂₄ fatty alcohols comprising from 1 to 200 ethylene oxide groups and (C₆-C₂₄ alkyl)polyglycosides.

When the composition comprises one or more surfactant(s), the total content of surfactant(s) in the composition preferably varies from 0.01% to 30% by weight, more preferentially from 0.1% to 20% by weight, better still from 0.5% to 15% by weight, even better still from 1% to 10% by weight, with respect to the total weight of the composition.

When the composition comprises one or more nonionic surfactant(s), the total content of nonionic surfactant(s) in the composition preferably varies from 0.01% to 30% by weight, more preferentially from 0.1% to 20% by weight, better still from 0.5% to 15% by weight, even better still from 1% to 10% by weight, with respect to the total weight of the composition.

### Fatty Substance(s)

The composition according to the invention can comprise one or more fatty substances.

The term “fatty substance” is understood to mean an organic compound which is insoluble in water at 25° C. and at atmospheric pressure (1.013×10⁵ Pa) (solubility of less than 5% by weight, preferably of less than 1% by weight, more preferentially still of less than 0.1% by weight). They exhibit in their structure at least one hydrocarbon chain comprising at least 6 carbon atoms and/or a sequence of at least two siloxane groups. In addition, the fatty substances are generally soluble in organic solvents under the same temperature and pressure conditions, such as, for example, chloroform, dichloromethane, carbon tetrachloride, ethanol, benzene, toluene, tetrahydrofuran (THF), liquid petrolatum or decamethylcyclopentasiloxane.

Advantageously, the fatty substances which can be used in the present invention are neither (poly)oxyalkylenated nor (poly)glycerolated.

Preferably, the fatty substances of use according to the invention are nonsilicone fatty substances.

The term “nonsilicone fatty substance” is understood to mean a fatty substance not containing Si—O bonds and the term “silicone fatty substance” is understood to mean a fatty substance containing at least one Si—O bond.

The fatty substances of use according to the invention can be liquid fatty substances (or oils) and/or solid fatty substances. Liquid fatty substance is understood to mean a fatty substance having a melting point of less than or equal to 25° C. at atmospheric pressure (1.013×10⁵ Pa). Solid fatty substance is understood to mean a fatty substance having a melting point of greater than 25° C. at atmospheric pressure (1.013×10⁵ Pa).

Within the meaning of the present invention, the melting point corresponds to the temperature of the most endothermic peak observed in thermal analysis (differential scanning calorimetry or DSC), as described in the standard ISO 11357-3:1999. The melting point can be measured using a differential scanning calorimeter (DSC), for example the calorimeter sold under the name MDSC 2920 by TA Instruments. In the present patent application, all the melting points are determined at atmospheric pressure (1.013×10⁵ Pa).

More particularly, the liquid fatty substance(s) according to the invention is/are chosen from C₆ to C₁₆ liquid hydrocarbons, liquid hydrocarbons comprising more than 16 carbon atoms, nonsilicone oils of animal origin, oils of triglyceride type of vegetable or synthetic origin, fluorinated oils, liquid fatty alcohols, liquid esters of fatty acid and/or of fatty alcohol other than the triglycerides, silicone oils and their mixtures.

It should be remembered that the fatty alcohols, esters and acids more particularly exhibit at least one saturated or unsaturated, linear or branched, hydrocarbon group comprising from 6 to 40, better still from 8 to 30, carbon atoms, which is optionally substituted, in particular by one or more hydroxyl groups (in particular 1 to 4). If they are unsaturated, these compounds can comprise from one to three conjugated or nonconjugated carbon-carbon double bonds.

As regards the C₆ to C₁₆ liquid hydrocarbons, the latter can be linear, branched or optionally cyclic and are preferably chosen from alkanes. Mention may be made, by way of example, of hexane, cyclohexane, undecane, dodecane, isododecane, tridecane or isoparaffins, such as isohexadecane or isodecane, and their mixtures.

The liquid hydrocarbons comprising more than 16 carbon atoms can be linear or branched and of mineral or synthetic origin and are preferably chosen from liquid paraffins or liquid petrolatum (INCI name: mineral oil or paraffinum liquidum), polydecenes, hydrogenated polyisobutene such as Parleam®, and their mixtures.

Mention may be made, as hydrocarbon oils of animal origin, of perhydrosqualene.

The triglyceride oils of vegetable or synthetic origin are preferably chosen from liquid triglycerides of fatty acids comprising from 6 to 30 carbon atoms, such as triglycerides of heptanoic or octanoic acid, or also, for example, sunflower oil, corn oil, soybean oil, marrow oil, grapeseed oil, sesame oil, hazelnut oil, apricot oil, macadamia oil, arara oil, castor oil, avocado oil, triglycerides of caprylic/capric acids, such as those sold by Stearinerie Dubois or those sold under the names Miglyol® 810, 812 and 818 by Dynamit Nobel, jojoba oil, shea butter oil and their mixtures.

As regards the fluorinated oils, the latter can be chosen from perfluoromethylcyclopentane and perfluoro-1,3-dimethylcyclohexane, sold under the names Flutec® PC1 and Flutec® PC3 by BNFL Fluorochemicals; perfluoro-1,2-dimethylcyclobutane; perfluoroalkanes, such as dodecafluoropentane and tetradecafluorohexane, sold under the names PF 5050® and PF 5060® by 3M, or also bromoperfluorooctyl, sold under the name Foralkyl® by Atochem; nonafluoromethoxybutane and nonafluoroethoxyisobutane; perfluoromorpholine derivatives, such as 4-trifluoromethylperfluoromorpholine sold under the name PF 5052® by 3M.

The liquid fatty alcohols suitable for the implementation of the invention are more particularly chosen from saturated or unsaturated and linear or branched alcohols, preferably unsaturated or branched alcohols, comprising from 6 to 40 carbon atoms, preferably from 8 to 30 carbon atoms. Mention may be made, for example, of octyldodecanol, 2-butyloctanol, 2-hexyldecanol, 2-undecylpentadecanol, isostearyl alcohol, oleyl alcohol, linolenyl alcohol, ricinoleyl alcohol, undecylenyl alcohol or linoleyl alcohol, and their mixtures.

As regards the liquid esters of fatty acid and/or of fatty alcohol other than the triglycerides mentioned above, mention may be made in particular of esters of saturated or unsaturated, linear C₁ to C₂₆ or branched C₃ to C₂₆, aliphatic mono- or polyacids and of saturated or unsaturated, linear C₁ to C₂₆ or branched C₃ to C₂₆, aliphatic mono- or polyalcohols, the total carbon number of the esters being greater than or equal to 6, more advantageously greater than or equal to 10.

Preferably, for the esters of monoalcohols, at least one of the alcohol or of the acid from which the esters of the invention result is branched.

Mention may be made, among the monoesters, of dihydroabietyl behenate; octyldodecyl behenate; isocetyl behenate; isostearyl lactate; lauryl lactate; linoleyl lactate; oleyl lactate; isostearyl octanoate; isocetyl octanoate; octyl octanoate; decyl oleate; isocetyl isostearate; isocetyl laurate; isocetyl stearate; isodecyl octanoate; isodecyl oleate; isononyl isononanoate; isostearyl palmitate; methyl acetyl ricinoleate; octyl isononanoate; 2-ethylhexyl isononanoate; octyldodecyl erucate; oleyl erucate; ethyl and isopropyl palmitates, such as 2-ethylhexyl palmitate or 2-octyldecyl palmitate; alkyl myristates, such as isopropyl myristate; isobutyl stearate; 2-hexyldecyl laurate, and their mixtures.

Use will preferably be made, among the monoesters of monoacids and of monoalcohols, of ethyl palmitate and isopropyl palmitate, alkyl myristates, such as isopropyl myristate or ethyl myristate, isocetyl stearate, 2-ethylhexyl isononanoate, isodecyl neopentanoate, isostearyl neopentanoate and their mixtures.

Still within the context of this alternative form, use may also be made of esters of C₄ to C₂₂ di- or tricarboxylic acids and of C₁ to C₂₂ alcohols and esters of mono-, di- or tricarboxylic acids and of di-, tri-, tetra- or pentahydroxy alcohols having from 2 to 26 carbon atoms.

Mention may in particular be made of: diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate; tridecyl erucate; triisopropyl citrate; triisostearyl citrate; glyceryl trilactate; glyceryl trioctanoate; trioctyldodecyl citrate; trioleyl citrate; propylene glycol dioctanoate; neopentyl glycol diheptanoate; diethylene glycol diisononanoate; polyethylene glycol distearates, and their mixtures.

The composition can also comprise, as fatty ester, sugar esters and diesters of fatty acids comprising from 6 to 30, preferably from 12 to 22, carbon atoms. It should be remembered that “sugar” is understood to mean oxygen-containing hydrocarbon compounds which have several alcohol functions, with or without an aldehyde or ketone function, and which comprise at least 4 carbon atoms. These sugars can be monosaccharides, oligosaccharides or polysaccharides.

Mention may be made, as suitable sugars, for example, of sucrose, glucose, galactose, ribose, fucose, maltose, fructose, mannose, arabinose, xylose, lactose and their derivatives, in particular alkylated derivatives, such as methylated derivatives, for example methylglucose.

The sugar esters of fatty acids can be chosen in particular from the group comprising the esters or mixtures of esters of sugars described above and of saturated or unsaturated and linear or branched C₆ to C₃₀, preferably C₁₂ to C₂₂, fatty acids. If they are unsaturated, these compounds can comprise from one to three conjugated or nonconjugated carbon-carbon double bonds.

The esters according to this alternative form can also be chosen from mono-, di-, tri- and tetraesters, polyesters and their mixtures.

These esters can, for example, be oleates, laurates, palmitates, myristates, behenates, cocoates, stearates, linoleates, linolenates, caprates, arachidonates or their mixtures, such as in particular the oleate/palmitate, oleate/stearate or palmitate/stearate mixed esters.

More particularly, use is made of mono- and diesters and in particular of sucrose, glucose or methylglucose mono- or dioleates, -stearates, -behenates, -oleate/palmitates, -linoleates, -linolenates or -oleate/stearates, and their mixtures.

Mention may be made, by way of example, of the product sold under the name Glucate® DO by Amerchol, which is a methylglucose dioleate.

Preferably, use will be made of a liquid ester of monoacid and of monoalcohol.

The silicone oils which can be used in the composition according to the present invention can be volatile or nonvolatile and cyclic, linear or branched silicone oils which are unmodified or modified by organic groups, and preferably have a viscosity of 5×10⁻⁶ to 2.5 m²/s at 25° C. and preferably of 1×10⁻⁵ to 1 m²/s.

Preferably, the silicone oils are chosen from polydialkylsiloxanes, in particular polydimethylsiloxanes (PDMS), and liquid polyorganosiloxanes comprising at least one aryl group.

These silicone oils can also be organomodified. The organomodified silicone oils which can be used in accordance with the invention are preferably liquid silicones as defined above and comprising in their structure one or more organofunctional groups attached via a hydrocarbon group, for example chosen from amine groups and alkoxy groups.

Organopolysiloxanes are defined in more detail in the work by Walter Noll, Chemistry and Technology of Silicones (1968), Academic Press. They may be volatile or nonvolatile.

When they are volatile, the silicone oils are more particularly chosen from those having a boiling point of between 60° C. and 260° C., and more particularly still from:

(i) cyclic polydialkylsiloxanes comprising from 3 to 7, preferably from 4 to 5, silicon atoms. These are, for example, octamethylcyclotetrasiloxane, sold in particular under the name Volatile Silicone® 7207 by Union Carbide or Silbione® 70045 V2 by Rhodia, decamethylcyclopentasiloxane, sold under the name Volatile Silicone® 7158 by Union Carbide and Silbione® 70045 V5 by Rhodia, and also their mixtures.

Mention may also be made of cyclocopolymers of the dimethylsiloxane/methylalkylsiloxane type, such as Volatile Silicone® FZ 3109 sold by Union Carbide.

Mention may also be made of mixtures of cyclic polydialkylsiloxanes with silicon-derived organic compounds, such as the mixture of octamethylcyclotetrasiloxane and of tetra(trimethylsilyl)pentaerythritol (50/50) and the mixture of octamethylcyclotetrasiloxane and of oxy-1,1′-bis(2,2,2′,2′,3,3′-hexatrimethylsilyloxy)neopentane;

(ii) volatile linear polydialkylsiloxanes having from 2 to 9 silicon atoms and exhibiting a viscosity of less than or equal to 5×10⁻⁶ m²/s at 25° C. It is, for example, decamethyltetrasiloxane, sold in particular under the name SH 200 by Toray Silicone. Silicones falling within this category are also described in the paper published in Cosmetics and Toiletries, Vol. 91, January 76, pages 27-32, Todd & Byers, Volatile Silicone Fluids for Cosmetics.

Use is preferably made of nonvolatile polydialkylsiloxanes.

These silicone oils are more particularly chosen from polydialkylsiloxanes, among which mention may be made mainly of polydimethylsiloxanes having trimethylsilyl end groups. The viscosity of the silicones is measured at 25° C. according to the standard ASTM D445 Appendix C.

Mention may be made, among these polydialkylsiloxanes, in a nonlimiting way, of the following commercial products:
the Silbione® oils of the 47 and 70 047 series or the Mirasil® oils sold by Rhodia, such as, for example, the oil 70 047 V 500 000;
the oils of the Mirasil® series sold by Rhodia;
the oils of the 200 series from Dow Corning, such as DC200 having a viscosity of 60 000 mm²/s;
the Viscasil® oils from General Electric and certain oils of the SF series (SF 96, SF 18) from General Electric.

Mention may also be made of polydimethylsiloxanes having dimethylsilanol end groups, known under the name dimethiconol (CTFA), such as the oils of the 48 series from Rhodia.

The organomodified silicones which can be used in accordance with the invention are silicones as defined above and comprising in their structure one or more organofunctional groups attached via a hydrocarbon group.

As regards the liquid polyorganosiloxanes comprising at least one aryl group, they can in particular be polydiphenylsiloxanes, and polyalkylarylsiloxanes functionalized by the abovementioned organofunctional groups.

The polyalkylarylsiloxanes are chosen particularly from linear and/or branched polydimethyl/methylphenylsiloxanes or polydimethyl/diphenylsiloxanes with a viscosity ranging from 1×10⁻⁵ to 5×10⁻² m²/s at 25° C.

Mention may be made, among these polyalkylarylsiloxanes, by way of example, of the products sold under the following names:
the Silbione® oils of the 70 641 series from Rhodia;
the oils of the Rhodorsil® 70 633 and 763 series from Rhodia;
the oil Dow Corning 556 Cosmetic Grade Fluid from Dow Corning;
the silicones of the PK series from Bayer, such as the product PK20;
the silicones of the PN and PH series from Bayer, such as the products PN1000 and PH1000;
certain oils of the SF series from General Electric, such as SF 1023, SF 1154, SF 1250 or SF 1265.

Mention may be made, among the organomodified silicones, of the polyorganosiloxanes comprising:
substituted or unsubstituted amine groups, such as the products sold under the names GP 4 Silicone Fluid and GP 7100 by Genesee or the products sold under the names Q2 8220 and Dow Corning 929 or 939 by Dow Corning. The substituted amine groups are in particular C₁ to C₄ aminoalkyl groups;
alkoxylated groups,
hydroxyl groups.

The solid fatty substances according to the invention preferably exhibit a viscosity of greater than 2 Pa·s, measured at 25° C. and at a shear rate of 1 s⁻¹.

The solid fatty substance(s) are preferably chosen from solid fatty acids, solid fatty alcohols, solid esters of fatty acids and/or of fatty alcohols, waxes, ceramides and their mixtures.

The term “fatty acid” is understood to mean a long-chain carboxylic acid comprising from 6 to 40 carbon atoms, preferably from 8 to 30 carbon atoms. The solid fatty acids according to the invention preferentially comprise from 10 to 30 carbon atoms and better still from 14 to 22 carbon atoms. They can optionally be hydroxylated. These fatty acids are neither oxyalkylenated nor glycerolated.

The solid fatty acids which can be used in the present invention are in particular chosen from myristic acid, cetylic acid, stearylic acid, palmitic acid, arachidic acid, stearic acid, lauric acid, behenic acid, 12-hydroxystearic acid and their mixtures.

Particularly preferably, the solid fatty acid(s) are chosen from stearic acid, myristic acid and palmitic acid.

The term “fatty alcohol” is understood to mean a long-chain aliphatic alcohol comprising from 6 to 40 carbon atoms, preferably from 8 to 30 carbon atoms, and comprising at least one hydroxyl OH group. These fatty alcohols are neither oxyalkylenated nor glycerolated.

The solid fatty alcohols can be saturated or unsaturated and linear or branched, and they comprise from 8 to 40 carbon atoms, preferably from 10 to 30 carbon atoms. Preferably, the solid fatty alcohols are of structure R—OH with R denoting a linear alkyl group, optionally substituted by one or more hydroxyl groups, comprising from 8 to 40, preferentially from 10 to 30, carbon atoms, better still from 10 to 30, indeed even from 12 to 24, carbon atoms, even better still from 14 to 22 carbon atoms.

The solid fatty alcohols capable of being used are preferably chosen from saturated or unsaturated, linear or branched, preferably linear and saturated, (mono)alcohols comprising from 8 to 40 carbon atoms, better still from 10 to 30, indeed even from 12 to 24, carbon atoms, even better still from 14 to 22 carbon atoms.

The solid fatty alcohols capable of being used can be chosen, alone or as a mixture, from: myristyl alcohol (or 1-tetradecanol); cetyl alcohol (or 1-hexadecanol); stearyl alcohol (or 1-octadecanol); arachidyl alcohol (or 1-eicosanol); behenyl alcohol (or 1-docosanol); lignoceryl alcohol (or 1-tetracosanol); ceryl alcohol (or 1-hexacosanol); montanyl alcohol (or 1-octacosanol); myricyl alcohol (or 1-triacontanol).

Preferentially, the solid fatty alcohol is chosen from cetyl alcohol, stearyl alcohol, behenyl alcohol, myristyl alcohol, arachidyl alcohol and their mixtures, such as cetylstearyl or cetearyl alcohol. Particularly preferably, the solid fatty alcohol is chosen from cetylstearyl or cetearyl alcohol and cetyl alcohol.

The solid esters of a fatty acid and/or of a fatty alcohol capable of being used are preferably chosen from esters resulting from a C₉-C₂₆ fatty carboxylic acid and/or from a C₉-C₂₆ fatty alcohol.

Preferably, these solid fatty esters are esters of a saturated, linear or branched, carboxylic acid comprising at least 10 carbon atoms, preferably from 10 to 30 carbon atoms and more particularly from 12 to 24 carbon atoms, and of a saturated, linear or branched, monoalcohol comprising at least 10 carbon atoms, preferably from 10 to 30 carbon atoms and more particularly from 12 to 24 carbon atoms. The saturated carboxylic acids can optionally be hydroxylated and are preferably monocarboxylic acids.

Use may also be made of esters of C₄-C₂₂ di- or tricarboxylic acids and of C₁-C₂₂ alcohols and of esters of mono-, di- or tricarboxylic acids and of C₂-C₂₆ di-, tri-, tetra- or pentahydroxy alcohols.

Mention may in particular be made of octyldodecyl behenate, isocetyl behenate, cetyl lactate, stearyl octanoate, octyl octanoate, cetyl octanoate, decyl oleate, hexyl stearate, octyl stearate, myristyl stearate, cetyl stearate, stearyl stearate, octyl pelargonate, cetyl myristate, myristyl myristate, stearyl myristate, diethyl sebacate, diisopropyl sebacate, diisopropyl adipate, di-n-propyl adipate, dioctyl adipate, dioctyl maleate, octyl palmitate, myristyl palmitate, cetyl palmitate, stearyl palmitate and their mixtures.

Preferably, the solid esters of a fatty acid and/or of a fatty alcohol are chosen from C₉-C₂₆ alkyl palmitates, in particular myristyl, cetyl or stearyl palmitate; C₉-C₂₆ alkyl myristates, such as cetyl myristate, stearyl myristate and myristyl myristate; and C₉-C₂₆ alkyl stearates, in particular myristyl, cetyl and stearyl stearate; and their mixtures.

Within the meaning of the present invention, a wax is a lipophilic compound, which is solid at 25° C. and atmospheric pressure, with a reversible solid/liquid change in state, having a melting point of greater than approximately 40° C. and which can range up to 200° C., and exhibiting, in the solid state, an anisotropic crystal organization. In general, the size of the crystals of the wax is such that the crystals diffract and/or scatter light, conferring on the composition which comprises them a cloudy, more or less opaque, appearance. On bringing the wax to its melting point, it is possible to render it miscible with oils and to form a microscopically homogeneous mixture but, on returning the temperature of the mixture to ambient temperature, recrystallization of the wax, which is detectable microscopically and macroscopically (opalescence), is obtained.

In particular, the waxes which are suitable for the invention can be chosen from waxes of animal, vegetable or mineral origin, synthetic nonsilicone waxes and their mixtures.

Mention may in particular be made of hydrocarbon waxes, such as beeswax, in particular of biological origin, lanolin wax and Chinese insect waxes; rice bran wax, carnauba wax, candelilla wax, ouricury wax, esparto grass wax, berry wax, shellac wax, Japan wax and sumac wax; montan wax, orange wax and lemon wax, microcrystalline waxes, paraffins and ozokerite; polyethylene waxes, the waxes obtained by the Fischer-Tropsch synthesis and waxy copolymers, and also their esters.

Mention may also be made of C₂₀ to C₆₀ microcrystalline waxes, such as Microwax HW.

Mention may also be made of the MW 500 polyethylene wax sold under the reference Permalen 50-L polyethylene.

Mention may also be made of the waxes obtained by catalytic hydrogenation of animal or vegetable oils having linear or branched C8 to C₃₂ fatty chains. Mention may in particular be made, among the latter, of isomerized jojoba oil, such as partially hydrogenated trans-isomerized jojoba oil, in particular the product manufactured or sold by Desert Whale under the commercial reference Iso-Jojoba-50®, hydrogenated sunflower oil, hydrogenated castor oil, hydrogenated copra oil, hydrogenated lanolin oil and di(1,1,1-trimethylolpropane) tetrastearate, in particular the product sold under the name Hest 2T-4S® by Heterene.

Use may also be made of the waxes obtained by hydrogenation of castor oil esterified with cetyl alcohol, such as those sold under the names Phytowax Castor 16L64® and 22L73® by Sophim.

Use may also be made, as wax, of a C₂₀ to C₄₀ alkyl (hydroxystearyloxy)stearate (the alkyl group comprising from 20 to 40 carbon atoms), alone or as a mixture. Such a wax is in particular sold under the names Kester Wax K 82 P®, Hydroxypolyester K 82 P® and Kester Wax K 80 P® by Koster Keunen.

It is also possible to use microwaxes in the compositions of the invention; mention may in particular be made of carnauba microwaxes, such as that sold under the name MicroCare 350® by Micro Powders, synthetic wax microwaxes, such as that sold under the name MicroEase 114S® by Micro Powders, microwaxes constituted of a mixture of carnauba wax and of polyethylene wax, such as those sold under the names Micro Care 300® and 310® by Micro Powders, microwaxes constituted of a mixture of carnauba wax and of synthetic wax, such as that sold under the name Micro Care 325® by Micro Powders, polyethylene microwaxes, such as those sold under the names Micropoly 200®, 220®, 220L® and 250S® by Micro Powders, and polytetrafluoroethylene microwaxes, such as those sold under the names Microslip 519® and 519 L® by Micro Powders.

The waxes are preferably chosen from mineral waxes, such as paraffin, petrolatum, lignite or ozokerite wax; vegetable waxes, such as cocoa butter or cork fiber or sugar cane waxes, olive tree wax, rice wax, hydrogenated jojoba wax, ouricury wax, carnauba wax, candelilla wax, esparto grass wax, or absolute waxes of flowers, such as the essential wax of blackcurrant blossom sold by Bertin (France); waxes of animal origin, such as beeswaxes or modified beeswaxes (cera bellina), spermaceti, lanolin wax and lanolin derivatives; microcrystalline waxes; and their mixtures.

Ceramides, or ceramide analogs, such as glycoceramides, capable of being used in the compositions according to the invention, are known; mention may in particular be made of ceramides of classes I, II, III and V according to the Dawning classification.

The ceramides or their analogs capable of being employed preferably correspond to the following formula: R³CH(OH)CH(CH₂OR²)(NHCOR¹), in which:
R¹ denotes a saturated or unsaturated, linear or branched, alkyl group derived from C₁₄-C₃₀ fatty acids, it being possible for this group to be substituted by a hydroxyl group in the α position, or a hydroxyl group in the ω position esterified by a saturated or unsaturated C₁₆-C₃₀ fatty acid;
R² denotes a hydrogen atom, a (glycosyl)ₙ group, a (galactosyl)ₘ group or a sulfogalactosyl group, in which n is an integer varying from 1 to 4 and m is an integer varying from 1 to 8;
R³ denotes a C₁₅-C₂₆ hydrocarbon group which is saturated or unsaturated in the α position, it being possible for this group to be substituted by one or more C₁-C₁₄ alkyl groups; it being understood that, in the case of natural ceramides or glycoceramides, R³ can also denote a C₁₅-C₂₆ α-hydroxyalkyl group, the hydroxyl group being optionally esterified by a C₁₆-C₃₀ α-hydroxy acid.

The ceramides which are more particularly preferred are the compounds for which R¹ denotes a saturated or unsaturated alkyl derived from C₁₆-C₂₂ fatty acids; R² denotes a hydrogen atom and R³ denotes a saturated linear C₁₅ group.

Preferentially, use is made of ceramides for which R¹ denotes a saturated or unsaturated alkyl group derived from C₁₄-C₃₀ fatty acids; R² denotes a galactosyl or sulfogalactosyl group; and R³ denotes a —CH═CH—(CH₂)₁₂—CH₃ group.

Use may also be made of the compounds for which R¹ denotes a saturated or unsaturated alkyl radical derived from C₁₂-C₂₂ fatty acids; R² denotes a galactosyl or sulfogalactosyl radical and R³ denotes a saturated or unsaturated C₁₂-C₂₂ hydrocarbon radical and preferably a —CH═CH—(CH₂)₁₂—CH₃ group.

Mention may also be made, as compounds which are particularly preferred, of 2-N-linoleoylaminooctadecane-1,3-diol; 2-N-oleoylaminooctadecane-1,3-diol; 2-N-palmitoylaminooctadecane-1,3-diol; 2-N-stearoylaminooctadecane-1,3-diol; 2-N-behenoylaminooctadecane-1,3-diol; 2-N-[2-hydroxypalmitoyl]aminooctadecane-1,3-diol; 2-N-stearoylaminooctadecane-1,3,4-triol and in particular N-stearoylphytosphingosine, 2-N-palmitoylaminohexadecane-1,3-diol, N-linoleoyldihydrosphingosine, N-oleoyldihydrosphingosine, N-palmitoyldihydrosphingosine, N-stearoyldihydrosphingosine, and N-behenoyldihydrosphingosine, N-docosanoyl-N-methyl-D-glucamine, cetylic acid N-(2-hydroxyethyl)-N-(3-cetyloxy-2-hydroxypropyl)amide and bis(N-hydroxyethyl-N-cetyl)malonamide; and their mixtures. Use will preferably be made of N-oleoyldihydrosphingosine.

The solid fatty substances are preferably chosen from solid fatty acids, solid fatty alcohols and their mixtures.

According to a preferred embodiment, the composition according to the invention comprises at least one liquid fatty substance, preferentially chosen from liquid hydrocarbons containing more than 16 carbon atoms, vegetable oils, liquid fatty alcohols, liquid fatty esters, silicone oils and their mixtures.

According to another particularly preferred embodiment, the composition according to the invention comprises at least one liquid fatty substance chosen from liquid hydrocarbons comprising more than 16 carbon atoms, in particular liquid petrolatum, liquid fatty alcohols, and their mixtures.

According to another preferred embodiment, the composition according to the invention comprises at least one solid fatty substance, preferentially chosen from solid fatty alcohols.

When the composition according to the invention comprises one or more fatty substances, the total content of the fatty substance(s) preferably varies from 5% to 80% by weight, more preferentially from 8% to 70% by weight and better still from 10% to 65% by weight, with respect to the total weight of the composition.

In a specific embodiment, the composition according to the invention comprises one or more fatty substances, the total content of the fatty substance(s) preferably varying from 30% to 80% by weight, more preferentially from 35% to 70% by weight and better still from 40% to 65% by weight, with respect to the total weight of the composition.

In another specific embodiment, the composition according to the invention comprises one or more liquid fatty substances, the total content of the liquid fatty substance(s) preferably varying from 30% to 80% by weight, more preferentially from 35% to 70% by weight and better still from 40% to 65% by weight, with respect to the total weight of the composition.

### Sequestering Agent

The composition according to the invention can comprise a sequestering agent (or chelating agent) other than etidronic acid, 2,6-dipicolinic acid and their salts.

The definition of a “sequestering agent” (or “chelating agent”) is well known to a person skilled in the art and refers to a compound or a mixture of compounds which is/are capable of forming a chelate with a metal ion. A chelate is an inorganic complex in which a compound (the sequestering or chelating agent) is coordinated to a metal ion, that is to say that it forms one or more bonds with the metal ion (formation of a ring including the metal ion).

A sequestering (or chelating) agent generally comprises at least two electron-donating atoms which make possible the formation of bonds with the metal ion.

Within the context of the present invention, the sequestering agent(s) can be chosen from carboxylic acids, preferably aminocarboxylic acids, phosphonic acids, preferably aminophosphonic acids, polyphosphoric acids, preferably linear polyphosphoric acids, their salts and derivatives.

The salts are in particular alkali metal, alkaline earth metal, ammonium and substituted ammonium salts.

Mention may be made, by way of example of sequestering agent based on carboxylic acids, of the following compounds: diethylenetriaminepentaacetic acid (DTPA), ethylenediaminedisuccinic acid (EDDS) and trisodium ethylenediaminedisuccinate, such as Octaquest E30 from Octel, ethylenediaminetetraacetic acid (EDTA) and its salts, such as disodium EDTA, tetrasodium EDTA, ethylenediamine-N,N′-diglutaric acid (EDDG), glycinamide-N,N′-disuccinic acid (GADS), 2-hydroxypropylenediamine-N,N′-disuccinic acid (HPDDS), ethylenediamine-N,N′-bis(ortho-hydroxyphenylacetic acid) (EDDHA), N,N′-bis(2-hydroxybenzyl)ethylenediamine-N,N′-diacetic acid (HBED), nitrilotriacetic acid (NTA), methylglycinediacetic acid (MGDA), N-2-hydroxyethyl-N,N-diacetic acid and glyceryliminodiacetic acid (as described in EP-A-317 542 and EP-A-399 133), iminodiacetic acid-N-2-hydroxypropylsulfonic acid and aspartic acid-N-carboxymethyl-N-2-hydroxypropyl-3-sulfonic acid (as described in EP-A-516 102), R-alanine-N,N′-diacetic acid, aspartic acid-N,N′-diacetic acid, aspartic acid-N-monoacetic acid (described in EP-A-509 382), chelating agents based on iminodisuccinic acid (IDSA) (as described in EP-A-509 382), ethanoldiglycine acid, phosphonobutanetricarboxylic acid, such as the compound sold by Bayer under the reference Bayhibit AM, N,N-dicarboxymethylglutamic acid and its salts, such as tetrasodium glutamate diacetate (GLDA), such as Dissolvine GL38 or 45S from AkzoNobel.

Mention may be made, by way of example of chelating agents based on mono- or polyphosphonic acid, of the following compounds: diethylenetriaminepenta(methylenephosphonic acid) (DTPMP), ethane-1-hydroxy-1,1,2-triphosphonic acid (E1 HTP), ethane-2-hydroxy-1,1,2-triphosphonic acid (E2HTP), ethane-1-hydroxy-1,1-diphosphonic acid (EHDP), ethane-1,1,2-triphosphonic acid (ETP), ethylenediaminetetramethylenephosphonic acid (EDTMP).

Mention may be made, by way of example of chelating agents based on polyphosphoric acid, of the following compounds: sodium tripolyphosphate (STP), tetrasodium diphosphate, hexametaphosphoric acid, sodium metaphosphate, phytic acid.

According to one embodiment, the sequestering agent(s) which are of use according to the invention are phosphorus-based sequestering agents, that is to say sequestering agents which comprise one or more phosphorus atoms, preferably at least two phosphorus atoms.

The phosphorus-based sequestering agent(s) used in the composition according to the invention are preferably chosen from:
inorganic phosphorus-based derivatives preferably chosen from alkali metal or alkaline earth metal, preferably alkali metal, phosphates and pyrophosphates, such as sodium pyrophosphate, potassium pyrophosphate, sodium pyrophosphate decahydrate; and alkali metal or alkaline earth metal, preferably alkali metal, polyphosphates, such as sodium hexametaphosphate, sodium polyphosphate, sodium tripolyphosphate, sodium trimetaphosphate; which are optionally hydrated, and their mixtures;
organic phosphorus-based derivatives, such as organic (poly)phosphates and (poly)phosphonates.

Preferably, the phosphorus-based sequestering agent(s) is/are chosen from linear or cyclic compounds comprising at least two phosphorus atoms bonded together covalently via at least one linker L comprising at least one oxygen atom and/or at least one carbon atom.

The phosphorus-based sequestering agent(s) can be chosen from inorganic phosphorus-based derivatives, preferably comprising at least two phosphorus atoms. More preferentially, the phosphorus-based sequestering agent(s) is/are chosen from alkali metal or alkaline earth metal pyrophosphates, better still from alkali metal pyrophosphates, in particular sodium pyrophosphate (also known as tetrasodium pyrophosphate).

The phosphorus-based sequestering agent(s) can be chosen from organic phosphorus-based derivatives, preferably comprising at least two phosphorus atoms.

Thus, preferably, the phosphorus-based sequestering agent(s) is/are chosen from alkali metal pyrophosphates, and/or their alkali metal salts, and a mixture of these compounds.

According to the present invention, the sequestering agents are preferably chosen from diethylenetriaminepentaacetic acid (DTPA) and its salts, diethylenediaminetetraacetic acid (EDTA) and its salts, ethylenediaminedisuccinic acid (EDDS) and its salts, N,N-dicarboxymethylglutamic acid and its salts (GLDA), and their mixtures.

Preference is given, among the salts of these compounds, to the alkali metal salts and in particular the sodium or potassium salts.

When the composition comprises one or more sequestering agents other than etidronic acid, 2,6-dipicolinic acid and their salts, the total content of the sequestering agent(s) other than etidronic acid, 2,6-dipicolinic acid and their salts preferably varies from 0.001% to 15% by weight, more preferentially from 0.005% to 10% by weight, better still from 0.01% to 8% by weight, even better still from 0.05% to 5% by weight, with respect to the total weight of the composition.

### Alkaline Agent

The composition according to the present invention can comprise one or more inorganic, organic or hybrid alkaline agent(s).

Preferably, the composition according to the present invention comprises one or more inorganic, organic or hybrid alkaline agent(s).

Within the meaning of the present invention, the terms “alkaline agent” and “basifying agent” are used without distinction.

The inorganic basifying agent(s) are preferably chosen from aqueous ammonia, alkali metal carbonates or bicarbonates, such as sodium (hydrogen)carbonate and potassium (hydrogen)carbonate, alkali metal or alkaline earth metal phosphates, such as sodium phosphates or potassium phosphates, sodium or potassium hydroxides, and their mixtures.

The organic basifying agent(s) are preferably chosen from alkanolamines, amino acids, organic amines, oxyethylenated and/or oxypropylenated ethylenediamines, 1,3-diaminopropane, 1,3-diamino-2-propanol, spermine, spermidine and their mixtures.

The term “alkanolamine” is understood to mean an organic amine comprising a primary, secondary or tertiary amine function and one or more linear or branched C₁-C₈ alkyl groups carrying one or more hydroxyl radicals.

Organic amines chosen from alkanolamines, such as mono-, di- or trialkanolamines comprising from one to three identical or different C₁-C₄ hydroxyalkyl radicals, are suitable in particular for the implementation of the invention.

In particular, the alkanolamine(s) are chosen from monoethanolamine (MEA), diethanolamine, triethanolamine, monoisopropanolamine, diisopropanolamine, N,N-dimethylethanolamine, 2-amino-2-methyl-1-propanol, triisopropanolamine, 2-amino-2-methyl-1,3-propanediol, 3-amino-1,2-propanediol, 3-dimethylamino-1,2-propanediol, tris(hydroxymethyl)aminomethane and their mixtures.

Advantageously, the amino acids are basic amino acids comprising an additional amine function. Such basic amino acids are preferably chosen from histidine, lysine, arginine, ornithine or citrulline.

The organic amine can also be chosen from organic amines of heterocyclic type. Mention may in particular be made, besides histidine already mentioned in the amino acids, of pyridine, piperidine, imidazole, triazole, tetrazole or benzimidazole. The organic amine can also be chosen from amino acid dipeptides. Mention may in particular be made, as amino acid dipeptides which can be used in the present invention, of carnosine, anserine and balenine. The organic amine can also be chosen from compounds comprising a guanidine function. Mention may in particular be made, as amines of this type other than arginine which can be used in the present invention, of creatine, creatinine, 1,1-dimethylguanidine, 1,1-diethylguanidine, glycocyamine, metformin, agmatine, N-amidinoalanine, 3-guanidinopropionic acid, 4-guanidinobutyric acid and 2-([amino(imino)methyl]amino)ethane-1-sulfonic acid.

Use may in particular be made, as hybrid compounds, of guanidine carbonate or monoethanolamine hydrochloride.

The alkaline agent(s) of use according to the invention is/are preferably chosen from alkanolamines, such as monoethanolamine, diethanolamine or triethanolamine, aqueous ammonia, carbonates or bicarbonates, such as sodium (hydrogen)carbonate and potassium (hydrogen)carbonate, and their mixtures, more preferentially from aqueous ammonia and alkanolamines, better still from alkanolamines.

When the composition comprises at least one alkaline agent, the total content of the alkaline agent(s) preferably varies from 0.1% to 40% by weight, more preferentially from 0.5% to 30% by weight, better still from 1% to 20% by weight, even better still from 2% to 10% by weight, with respect to the total weight of the composition.

According to one embodiment, the pH of the composition comprising at least one alkaline agent is of between 8 and 13, preferably between 9 and 12.

The pH of the composition can be adjusted to the desired value by means of acidic or alkaline agent(s) commonly used in the dyeing of keratin fibers, such as those described above, or else using buffer systems known to a person skilled in the art.

### Oxidizing Agent

The composition according to the invention can comprise one or more chemical oxidizing agents.

Preferably, the chemical oxidizing agent(s) are chosen from hydrogen peroxide, urea peroxide, alkali metal bromates or ferricyanides, peroxygenated salts, such as, for example, persulfates, perborates, peracids and their precursors and percarbonates of alkali metals or alkaline earth metals and their mixtures. Preferably, the oxidizing agent is chosen from hydrogen peroxide.

When it is present, the total content of oxidizing agent, preferably of hydrogen peroxide, preferably ranges from 0.1% to 30% by weight, more preferentially from 1% to 20% by weight, better still from 2% to 10% by weight, with respect to the total weight of the composition.

### Solvents

The composition according to the invention can also comprise at least one organic solvent.

Mention may be made, for example, as organic solvent, of linear or branched C₂ to C₄ alkanols, such as ethanol and isopropanol; glycerol; polyols and polyol ethers, such as 2-butoxyethanol, propylene glycol, dipropylene glycol, propylene glycol monomethyl ether, diethylene glycol monoethyl ether and monomethyl ether, and also aromatic alcohols or ethers, such as benzyl alcohol or phenoxyethanol, and their mixtures.

The organic solvent(s) can be present in an amount ranging from 0.01% to 30% by weight, preferably ranging from 2% to 25% by weight, with respect to the total weight of the composition.

In addition, preferably, the composition according to the invention is an aqueous composition. Preferably, the composition comprises water in an amount of greater than or equal to 5% by weight, preferably of greater than or equal to 10% by weight, better still of greater than or equal to 15% by weight, with respect to the total weight of the composition.

### Additives

The composition according to the invention can optionally comprise one or more additives, other than the compounds of the invention, and among which mention may be made of cationic, anionic, nonionic or amphoteric polymers or their mixtures, inorganic thickening agents, antidandruff agents, antiseborrhoeic agents, agents for combating hair loss and/or for promoting hair growth, vitamins and provitamins including panthenol, sunscreens, inorganic or organic pigments, plasticizing agents, solubilizing agents, opacifying or pearlescent agents, antioxidants, hydroxy acids, fragrances or preserving agents.

Of course, a person skilled in the art will take care to choose this or these optional additional compounds such that the advantageous properties intrinsically attached to the composition according to the invention are not, or not substantially, detrimentally affected by the envisaged addition(s).

The above additives can generally be present in an amount, for each of them, of between 0% and 20% by weight, with respect to the total weight of the composition.

According to a specific embodiment, the composition according to the invention comprises:
at least one oxidation base chosen from 2-methoxymethyl-para-phenylenediamine of formula (I), one of its addition salts, its solvates and/or the solvates of its salts:

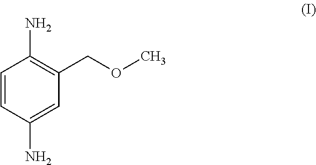

at least one oxidation coupler chosen from 6-hydroxybenzomorpholine of formula (II), its addition salts, its solvates and/or the solvates of its salts, hydroxyethyl-3,4-methylenedioxyaniline of formula (III), its addition salts, its solvates and/or the solvates of its salts, 2-amino-5-ethylphenol of formula (IV), its addition salts, its solvates and/or the solvates of its salts, and their mixtures:

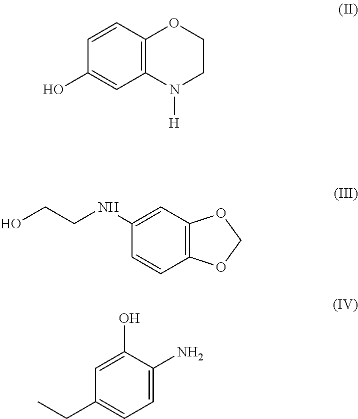

at least one compound chosen from etidronic acid, its salts and their mixtures, and
at least one compound chosen from 2,6-dipicolinic acid, its salts and their mixtures.

According to another specific embodiment, the composition according to the invention comprises:
at least one oxidation base chosen from 2-methoxymethyl-para-phenylenediamine of formula (I), one of its addition salts, its solvates and/or the solvates of its salts:

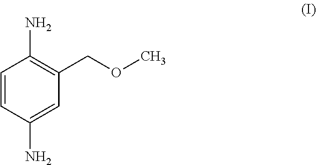

at least one oxidation coupler chosen from 6-hydroxybenzomorpholine of formula (II), its addition salts, its solvates and/or the solvates of its salts, hydroxyethyl-3,4-methylenedioxyaniline of formula (III), its addition salts, its solvates and/or the solvates of its salts, 2-amino-5-ethylphenol of formula (IV), its addition salts, its solvates and/or the solvates of its salts, and their mixtures:

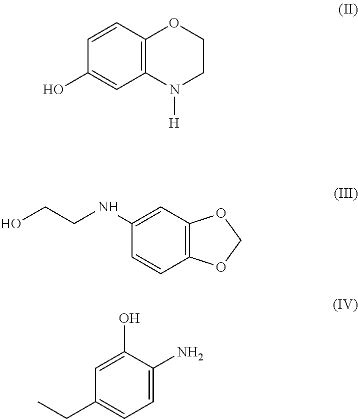

at least one compound chosen from etidronic acid, its salts and their mixtures,
at least one compound chosen from 2,6-dipicolinic acid, its salts and their mixtures,
at least one alkaline agent and
at least one oxidizing agent.

According to another specific embodiment, the composition according to the invention comprises:
at least one oxidation base chosen from 2-methoxymethyl-para-phenylenediamine of formula (I), one of its addition salts, its solvates and/or the solvates of its salts:

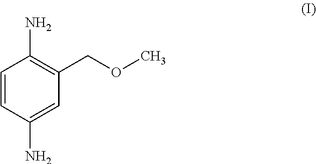

at least one oxidation coupler chosen from 6-hydroxybenzomorpholine of formula (II), its addition salts, its solvates and/or the solvates of its salts, hydroxyethyl-3,4-methylenedioxyaniline of formula (III), its addition salts, its solvates and/or the solvates of its salts, 2-amino-5-ethylphenol of formula (IV), its addition salts, its solvates and/or the solvates of its salts, and their mixtures:

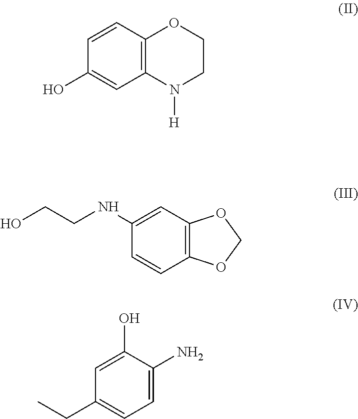

at least one compound chosen from etidronic acid, its salts and their mixtures,
at least one compound chosen from 2,6-dipicolinic acid, its salts and their mixtures,
at least one alkaline agent,
at least one oxidizing agent,
at least one surfactant, preferably a nonionic surfactant, and
at least one fatty substance.

### Method

The present invention also relates to a method for dyeing keratin fibers, preferably the hair, which comprises the stage of applying, to said keratin fibers, an effective amount of a composition as described above.

The composition can be applied to wet or dry keratin fibers. On conclusion of the treatment, the keratin fibers are optionally rinsed with water, optionally subjected to washing with a shampoo followed by rinsing with water, before being dried or left to dry. Preferably, the composition applied to the keratin fibers results from the mixing of at least two compositions:
a) a dyeing composition comprising:
   at least one oxidation base chosen from 2-methoxymethyl-para-phenylenediamine of formula (I), one of its addition salts, its solvates and/or the solvates of its salts:

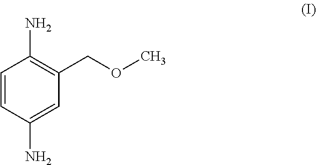

at least one oxidation coupler, and
optionally an alkaline agent as defined above,
b) an oxidizing composition comprising one or more chemical oxidizing agents such as those described above, preferably hydrogen peroxide,
the dyeing composition and/or the oxidizing composition comprising at least one compound chosen from etidronic acid, its salts and their mixtures and at least one compound chosen from 2,6-dipicolinic acid, its salts and their mixtures.

The dyeing method of the invention preferably comprises a stage of mixing a dyeing composition with an oxidizing composition comprising at least one chemical oxidizing agent. The mixing stage is preferably carried out at the time of use, immediately before the application of the composition according to the invention to the hair.

Preferably, at least one of the compositions (dyeing composition or oxidizing composition) is aqueous.

The dyeing composition can comprise one or more ingredients among those described above.

The oxidizing composition comprises an oxidizing agent as described above.

Preferably, the oxidizing composition comprises hydrogen peroxide as oxidizing agent, in aqueous solution, the concentration of which varies, more particularly, from 0.1% to 50%, more particularly between 0.5% and 20% and more preferentially still between 1% and 15% by weight, with respect to the weight of the oxidizing composition.

The oxidizing composition is preferably an aqueous composition. In particular, it comprises more than 5% by weight of water, preferably more than 10% by weight of water and more advantageously still more than 20% by weight of water.

It can also comprise one or more organic solvents chosen from those listed above; these solvents more particularly representing, when they are present, from 1% to 40% by weight and preferably from 5% to 30% by weight, with respect to the weight of the oxidizing composition.

Usually, the pH of the oxidizing composition, when it is aqueous, is less than 7.

The oxidizing composition can also preferably comprise one or more acidifying agents. Mention may be made, by way of example, among the acidifying agents, of inorganic or organic acids, such as hydrochloric acid, phosphoric acid, sulfuric acid, carboxylic acids, such as acetic acid, tartaric acid, citric acid or lactic acid, or sulfonic acids.

The oxidizing composition can comprise fatty substances, such as those described above, preferably chosen from fatty alcohols, liquid hydrocarbons comprising more than 16 carbon atoms and their mixtures, surfactants or polymers.

According to a preferred embodiment, the oxidizing composition comprises at least one compound chosen from etidronic acid, its salts and their mixtures and at least one compound chosen from 2,6-dipicolinic acid, its salts and their mixtures.

### Kit

Another subject matter of the invention is a device comprising at least two compartments for the dyeing of keratin fibers, at least a first compartment including the dyeing composition as described above and at least a second compartment including an oxidizing composition as described above.

The device according to the invention can also comprise a third compartment including, for example, a care composition.

The compositions of the device according to the invention are packaged in separate compartments, optionally accompanied by suitable application means, which are identical or different, such as fine brushes, coarse brushes or sponges.

The abovementioned device can also be equipped with a means making it possible to dispense the desired mixture over the hair, for example the devices described in the patent FR 2 586 913.

Finally, the present invention relates to the use of a composition as described above for the dyeing of keratin fibers and in particular of the hair.

The following examples serve to illustrate the invention without, however, exhibiting a limiting nature.

### EXAMPLES

In the examples which follow, all the amounts are shown as percentage by weight of active material (AM) with respect to the total weight of the composition (unless otherwise mentioned).

### Example 1

### Dyeing Composition

The composition A according to the present invention and the comparative composition C were prepared from the ingredients, the contents of which are shown in the table below:

TABLE 1

| | A | C |
|---|---|---|
| | (invention) | (comparative) |
| 2-Methoxymethyl-p-phenylenediamine | 2 mmol | — |
| Hydroxyethyl-p-phenylenediamine sulfate | | 2 mmol |
| Hydroxybenzomorpholine | 0.30 | 0.30 |
| Ethanolamine | 4.14 | 4.14 |
| Etidronic acid | 0.14 | 0.14 |
| 2,6-Dicarboxypyridine | 1.10 | 1.10 |
| Sodium metabisulfite | 0.22 | 0.22 |
| Sodium lauryl sulfate | 1.24 | 1.24 |
| Cocoyl glucoside | 3.02 | 3.02 |
| Hydroxypropyl guar | 1.00 | 1.00 |
| PEG-40 hydrogenated castor oil | 1.00 | 1.00 |
| Mineral oil | 60.00 | 60.00 |
| Water | q.s. 100 | q.s. 100 |

### Oxidizing Composition

The oxidizing composition B was prepared from the ingredients, the contents of which are shown in the table below:

TABLE 2

| | B |
|---|---|
| Hydrogen peroxide | 6.00 |
| Cetearyl alcohol | 6.00 |
| Mineral oil | 20.00 |
| Steareth-20 | 5.00 |
| Polyquaternium-6 | 0.20 |
| Hexadimethrine chloride | 0.15 |
| Tetrasodium etidronate | 0.06 |
| Tetrasodium pyrophosphate | 0.04 |
| Sodium salicylate | 0.035 |
| Glycerol | 0.50 |
| PEG-4 Rapeseedamide | 1.20 |
| Tocopherol | 0.10 |
| Phosphoric acid | q.s. pH 2.2 |
| Water | q.s. 100 |

### Dyeing Protocol

The dyeing compositions A and C are each mixed with the oxidizing composition B in a 1+1 ratio by weight.

Each of the mixtures is applied to locks of natural hair containing 90% of gray hairs, in a proportion of 5 g of mixture per 1 g of hair.

After a leave-on time of 35 minutes on a heating plate at 27° C., the hair is rinsed, washed with a standard shampoo and dried.

### Results

The coloring of the hair is evaluated in the L*a*b* system, with a Konica Minolta CM-3600A spectrocolorimeter (illuminant D65, angle 10°, specular component included) in the CIELab system.

In this system, L* represents the lightness. The lower the value of L*, the darker and more powerful the coloring obtained. The chromaticity C* is calculated from the values a* and b*, a* representing the red/green axis and b* the yellow/blue axis

C*=(a*²+b*²)^{1/2}.

The higher the value of C*, the better the chromaticity.

The results are shown in the table below:

TABLE 3

| | a* | b* | C* |
|---|---|---|---|
| A + B (invention) | 7.67 | 17.25 | 18.88 |
| C + B (comparative) | 2.99 | 13.23 | 13.56 |

The composition A according to the invention results in a higher C* value, and thus in a better chromaticity, with respect to the comparative composition C.

### Example 2

### Dyeing Composition

The composition A1 according to the present invention and the comparative composition C1 were prepared from the ingredients, the contents of which are shown in the table below:

TABLE 4

| | A1 | C1 |
|---|---|---|
| | (invention) | (comparative) |
| 2-METHOXYMETHYL-P- | 0.30 | 0.30 |
| PHENYLENEDIAMINE | | |
| HYDROXYBENZOMORPHOLINE | 0.30 | 0.30 |
| ETHANOLAMINE | 4.14 | 4.14 |
| ETIDRONIC ACID | 0.14 | 0.14 |
| 2,6-DICARBOXYPYRIDINE | 0.10 | — |
| ACIDE SALICYLIQUE | — | 0.10 |
| SODIUM METABISULFITE | 0.22 | 0.22 |
| SODIUM LAURYL SULFATE | 1.24 | 1.24 |
| COCO-GLUCOSIDE | 3.02 | 3.02 |
| HYDROXYPROPYL GUAR | 1.00 | 1.00 |
| PEG-40 HYDROGENATED | 1.00 | 1.00 |
| CASTOR OIL | | |
| MINERAL OIL/PARAFFINUM | 60.00 | 60.00 |
| LIQUIDUM | | |
| WATER | Qs 100 | Qs 100 |
| pH | 11 ± 0.2 | 11 ± 0.2 |

### Dyeing Protocol

The dyeing compositions A1 and C1 are each mixed with the oxidizing composition B in a 1+1 ratio by weight. Mixtures A1+B and C1+B have a pH equal to 10.1±0.2.

Each of the mixtures is applied to locks of natural hair containing 90% of grey hair (NG) and to locks of permanent-waved hair containing 90% of grey hair (PG) in a proportion of 5 g of mixture per 1 g of hair.

After a leave-on time of 35 minutes on a heating plate at 27° C., the hair is rinsed, washed with a standard shampoo and dried.

### Results

The colouring of the hair is evaluated in the L*a*b* system, using a Konica Minolta CM-3600A spectrocolorimeter (illuminant D65, angle 10°, specular component included) in the CIELab system.

In this system, L* represents the lightness. The lower the value of L*, the darker and more powerful the colouring obtained. The selectivity is represented by the color difference ΔE between the natural colored locks (NG) and the permed colored locks (PG). The lower the value of ΔE, the lower the selectivity, therefore the better.

The results are indicated in the table below:

TABLE 5

| | | L* | a* | b* | ΔΕ |
|---|---|---|---|---|---|
| A1 + B (invention) | NG | 34.66 | 6.24 | 14.83 | 6.52 |
| | PG | 28.81 | 6.22 | 11.95 | |
| C1 + B (comparative) | NG | 39.48 | 4.86 | 14.81 | 8.85 |
| | PG | 31.01 | 5.96 | 12.48 | |

Composition A1 according to the invention leads to a lower value of L*, and thus to better strength of the colour, compared to comparative composition C1.

Composition A1 according to the invention also leads to a lower value of ΔE, therefore to better selectivity, compared to comparative composition C1.

1-16. (canceled)

17. A composition comprising:
at least one oxidation base chosen from 2-methoxymethyl-para-phenylenediamine of formula (I), addition salts thereof, solvates thereof, solvates of salts thereof, or mixtures of two or more thereof: 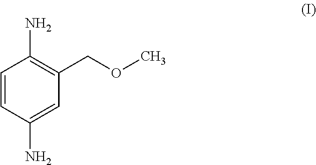
at least one oxidation coupler,
at least one compound chosen from etidronic acid, salts thereof, or mixtures of two or more thereof, and
at least one compound chosen from 2,6-dipicolinic acid, salts thereof, or mixtures of two or more thereof.

18. The composition of claim 17, wherein the total amount of oxidation base(s) ranges from 0.001% to 20% by weight, relative to the total weight of the composition.

19. The composition of claim 17, wherein the at least one oxidation coupler is chosen from 6-hydroxybenzomorpholine, hydroxyethyl-3,4-methylenedioxyaniline, 2-amino-5-ethylphenol, addition salts thereof, solvates thereof, solvates of salts thereof, or mixtures of two or more thereof.

20. The composition of claim 17, wherein the total amount of oxidation coupler(s) ranges from 0.001% to 20% by weight, relative to the total weight of the composition.

21. The composition of claim 17, wherein the total amount of compound(s) chosen from etidronic acid, salts thereof, or mixtures of two or more thereof ranges from 0.001% to 15% by weight, relative to the total weight of the composition.

22. The composition of claim 17, wherein the total amount of compound(s) chosen from 2,6-dipicolinic acid, salts thereof, or mixtures of two or more thereof ranges from 0.001% to 15% by weight, relative to the total weight of the composition.

23. The composition of claim 17, further comprises at least one fatty substance.

24. The composition of claim 17, further comprises at least one liquid fatty substance.

25. The composition of claim 17, further comprises at least one solid fatty substance.

26. The composition of claim 17, further comprises at least one surfactant chosen from anionic surfactants, nonionic surfactants, amphoteric surfactants, or mixtures of two or more thereof.

27. The composition of claim 17, further comprises at least one alkaline agent.

28. The composition of claim 17, further comprises at least one chemical oxidizing agent.

29. A method for dyeing keratin fibers, comprising applying to the keratin fibers, a mixture comprising a dyeing composition and an oxidizing composition,
wherein the dyeing composition comprises:
at least one oxidation base chosen from 2-methoxymethyl-para-phenylenediamine of formula (I), addition salts thereof, solvates thereof, solvates of salts thereof, or mixtures of two or more thereof: 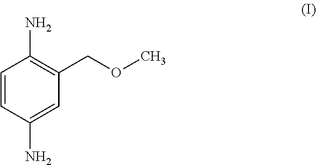
at least one oxidation coupler, and
optionally at least one alkaline agent;
wherein the oxidizing composition comprises at least one chemical oxidizing agent,
wherein at least one of the dyeing composition or the oxidizing composition comprises:
at least one compound chosen from etidronic acid, salts thereof, or mixtures of two or more thereof;
at least one compound chosen from 2,6-dipicolinic acid, salts thereof, or mixtures of two or more thereof; and
optionally at least one compound chosen from liquid fatty substances, solid fatty substances, at least one surfactant, or mixtures of two or more thereof.

30. The method of claim 29, wherein the dyeing composition comprises:
at least one compound chosen from etidronic acid, salts thereof, or mixtures of two or more thereof;
at least one compound chosen from 2,6-dipicolinic acid, salts thereof, or mixtures of two or more thereof; and
optionally at least one compound chosen from liquid fatty substances, solid fatty substances, at least one surfactant, or mixtures of two or more thereof.

31. The method of claim 29, wherein the at least one alkaline agent is chosen from alkanolamines.

32. The method of claim 29, wherein the liquid fatty substances are chosen from liquid hydrocarbons comprising more than 16 carbon atoms, vegetable oils, liquid fatty alcohols, liquid fatty esters, silicone oils, or mixtures of two or more thereof.

33. The method of claim 29, wherein the solid fatty substances are chosen from solid fatty acids, solid fatty alcohols, solid esters of fatty acids and/or of fatty alcohols, waxes, ceramides, or mixtures of two or more thereof.

34. The method of claim 29, wherein the least one surfactant is chosen from anionic surfactants, nonionic surfactants, amphoteric surfactants, or mixtures of two or more thereof.

35. The method of claim 29, wherein the at least one surfactant is chosen from nonionic surfactants.

36. A device for dyeing keratin fibers comprising:
at least one first compartment comprising a dyeing composition, and
at least one second compartment comprising an oxidizing composition,
wherein the dyeing composition comprises:
at least one oxidation base chosen from 2-methoxymethyl-para-phenylenediamine of formula (I), addition salts thereof, solvates thereof, solvates of salts thereof, or mixtures of two or more thereof: 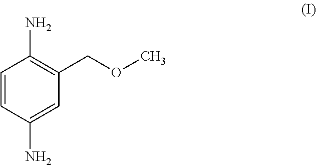
at least one oxidation coupler, and
optionally at least one alkaline agent,
wherein the oxidizing composition comprises at least one chemical oxidizing agent.
wherein at least one of the dyeing composition or the oxidizing composition comprises:
at least one compound chosen from etidronic acid, salts thereof, or mixtures of two or more thereof;
at least one compound chosen from 2,6-dipicolinic acid, salts thereof, or mixtures of two or more thereof; and
optionally at least one compound chosen from liquid fatty substances, solid fatty substances, at least one surfactant, or mixtures of two or more thereof.
